(19) United States
(12) Patent Application Publication
(10) Pub. No.: US 2023/0073951 A1
(43) Pub. Date: Mar. 9, 2023

(54) INSPECTION ASSISTANCE METHOD, INSPECTION ASSISTANCE DEVICE, INSPECTION ASSISTANCE SYSTEM, AND RECORDING MEDIUM

(71) Applicant: Evident Corporation, Nagano (JP)

(72) Inventor: Osamu MITSUNAGA, Kokubunji-shi (JP)

(73) Assignee: Evident Corporation, Nagano (JP)

(21) Appl. No.: 17/860,660

(22) Filed: Jul. 8, 2022

(30) Foreign Application Priority Data
Sep. 8, 2021 (JP) 2021-146215

(51) Int. Cl. A61B 1/00; A61B 1/045

(52) U.S. Cl. CPC A61B 1/00055; A61B 1/000094; A61B 1/045

(57) ABSTRACT

In an inspection assistance method, a processor accepts inspection portion information related to an inspection portion. In the inspection assistance method, the processor acquires a result of an inspection of the inspection portion corresponding to the inspection portion information from a storage media: In the inspection assistance method, the processor displays inspection assistance information on a display. The inspection assistance information notifies a user of information related to an abnormality of the inspection portion detected in a previous inspection. In the inspection assistance method, the processor displays an image of the inspection portion on the display.

### BACKGROUND OF THE INVENTION

### Field of the Invention

The present invention relates to an inspection assistance method, an inspection assistance device, an inspection assistance system, and a recording medium,

Priority is claimed on Japanese Patent Application No. 2021-146215, filed on Sep. 8, 2021, the content of which is incorporated herein by reference.

### Description of Related Art

Industrial endoscope devices have been used for an inspection of internal abnormalities, corrosion, and the like of boilers, pipes, automobile engines, and the like.

A user inspects an inspection portion in an inspection target by using an endoscope device. The user checks in an inspection whether abnormalities such as damage have occurred in the inspection portion. An image (a still image or a video) of the inspection portion is recorded in order to cord proof of the inspection. In a case in which an abnormality is found, information indicating the type and state of the abnormality is also recorded.

An inspection target includes so many inspection portions, and the type and state of an abnormality are different between inspection portions. Therefore, an inspector requires knowledge or skill from an inspection perspective, a possibility of an abnormality in each inspection portion, and the like. For example, in a case in which an inexperienced inspector performs an inspection, there is a possibility that an abnormality is overlooked.

A technique disclosed in Japanese Unexamined Patent Application, First Publication No. 2018-166959 provides a method of encouraging a user to pay attention to an image. When a still image is recorded, the user selects the type and state of an abnormality. Information indicating the type and state selected by the user is recorded along, with the still image. In addition, an inspection history table including a result of a previous inspection is updated. When the amount of an inspection result indicating an.

abnormality of an inspection target in the inspection history meets a predetermined condition, the user is encouraged to pay attention to an image.

### SUMMARY OF THE INVENTION

According to a first aspect of the present invention, an inspection assistance method is executed by a processor. The method includes accepting inspection portion information related to an inspection portion included in at least part of an inspection target. The method includes connecting to a storage medium storing a result of a previous inspection. The method includes acquiring the result of the inspection portion corresponding to the inspection portion information from the storage medium. The method includes displaying inspection assistance information on a display on the basis of the acquired result. The inspection assistance information notifies a user of information related to an abnormality of the inspection portion detected in the previous inspection. The method includes displaying an image of the inspection portion on the display after the inspection assistance information is displayed.

According to a second aspect of the present invention, in the first aspect, when two or more of the results are acquired, the displaying of the inspection assistance information may include displaying the inspection assistance information on the display on the basis of priority set in advance.

According to a third aspect of the present invention, in the second aspect, the priority may be set in accordance with a state of the abnormality.

According to a fourth aspect of the present invention, in the third aspect, the displaying of the inspection assistance information may include displaying the inspection assistance information on the display on the basis of the result indicating the state haying first priority. The displaying of the inspection assistance information may include displaying the inspection assistance information on the display on the basis of the result indicating the state having second priority lower than the first priority after the inspection assistance information is displayed on the basis of the result indicating the state having the first priority.

According to a fifth aspect of the present invention, in the second aspect, the priority may be set in accordance with the number of the abnormalities for each type of the abnormalities.

According to a sixth aspect of the present invention, in the fifth aspect, he displaying of the inspection assistance information may include displaying the inspection assistance information on the display on the basis of the result indicating the type of the abnormality detected a first number of times. The displaying of the inspection assistance information may include displaying the inspection assistance information the display on the basis of the result, indicating the type of the abnormality detected a second number of times after the inspection assistance information is displayed on the basis of the result indicating the type of the abnormality detected the first number of times. The second number is less than the first number.

According to a seventh aspect of the present invention, in the first aspect, the inspection assistance method may further include displaying the inspection portion information on the display before the inspection portion information is accepted.

According to an eighth aspect of the present invention, in the seventh aspect, the displaying of the inspection portion information may include referring to an inspection plan including the inspection portion information related to the inspection portion scheduled to be inspected. The displaying of the inspection portion information may include displaying the inspection portion information on the display on the basis of the inspection plan.

According to a ninth aspect of the present invention, in the seventh aspect, the displaying of the inspection portion information may include displaying the image of the inspection target on the display. The displaying of the inspection portion information may include displaying the inspection portion information on the image.

According to a tenth aspect of the present invention, in the first aspect, the inspection portion information may indicate a folder of the storage medium in which the result is saved. The folder may be prepared for each inspection portion and may be associated with the inspection portion. The acquiring of the result of the inspection portion may include acquiring the result of the inspection portion from the folder indicated by the inspection portion information.

According to an eleventh aspect of the present invention, in the first aspect, the inspection assistance information may include an image of the abnormality detected in a previous inspection.

According to a twelfth aspect of the present invention, in the first aspect, the inspection assistance information may indicate the number of the abnormalities for each type of the abnormalities.

According to a thirteenth aspect of the present invention, the first aspect, the inspection assistance information may indicate a proportion of the number of the abnormalities previously detected in the inspection portion for each type of the abnormalities to the number of all abnormalities previously detected in the inspection portion.

According to a fourteenth aspect of the present invention, in the first aspect, the acquiring of the result of the inspection portion may include acquiring the result of an inspection previously performed for the same object as the inspection target or for an object of the same type as a type of the inspection target.

According to a fifteenth aspect of the present invention, in the first aspect, the acquiring of the result of the inspection portion may include searching for the result of the inspection portion in the same object as the inspection target. The acquiring of the result of the inspection portion may include acquiring the result of the inspection portion in an object of the same type as a type of the inspection target in a case in which the result of the inspection portion is not acquired.

According to a sixteenth aspect of the present invention, in the first aspect, the displaying of the inspection assistance information may include displaying one of two or more sequentially generated images if the inspection portion on the display.

According to a seventeenth aspect of the present invention, in the first aspect, the inspection assistance method may further include after the inspection assistance information is displayed, hiding the inspection assistance information or reducing a size of a region of the inspection assistance information displayed on the display when movement of an insertion unit inserted into the inspection target. is detected.

According to an eighteenth aspect of the present invention, in the first aspect, the displaying of the inspection assistance information may include displaying the inspection assistance information on the image of the inspection portion. The displaying of the inspection assistance information may include, after the inspection assistance information is displayed, changing a transmittance of the inspection assistance information when movement of an insertion unit inserted into the inspection target is detected.

According to a nineteenth aspect of the present invention, an inspection assistance device includes a processor. The processor is configured to accept inspection portion information related to an inspection portion included in at least part of an inspection target. The processor is configured to connect to a storage medium storing a result of a previous inspection. The processor is configured to acquire the result of the inspection portion corresponding to the inspection portion information from the storage medium. The processor is configured to display inspection assistance information on a display on the basis of the acquired result. The inspection assistance information notifies a user of information related to an abnormality of the inspection portion detected in the previous inspection. The processor configured to display an image of the inspection portion on the display after the inspection assistance information is displayed.

According to a twentieth aspect of the present invention, an inspection assistance system includes an inspection assistance device and a server. The inspection assistance device includes a first communicator and a first processor. The first communicator is configured to transmit inspection portion information related to an inspection portion included in at least part of an inspection target to a server and receive a result of a previous inspection of the inspection portion from the server. The first processor is configured to accept the inspection portion information. The first processor is configured to display inspection assistance information on a display on the basis of the result received by the first communicator. The inspection assistance information notifies a user of information related to an abnormality of the inspection portion detected in the previous inspection. The first processor s configured to display an image of the inspection portion on the display after the inspection assistance information is displayed. The server includes a second processor and a second communicator. The second processor is configured to connect to a storage medium storing a result of a previous inspection and acquire the result of the inspection of the inspection portion corresponding to the inspection portion information from the storage medium. The second communicator is configured to receive the inspection portion information from the inspection assistance device and transmit the result acquired by the second processor to the inspection assistance device.

According to a twenty-first aspect of the present invention, a non-transitory computer-readable recording medium stores a program causing a computer to execute processing. The computer accepts inspection portion information related to an inspection portion included in at least part of an inspection target. The computer connects to a storage medium storing a result of a previous inspection and acquires the result of the inspection portion corresponding to the inspection portion information from the storage medium. The computer displays inspection assistance information on a display on the basis of the acquired result. The inspection assistance information notifies a user of information elated to an abnormality of the inspection portion detected in the previous inspection. The computer displays an image of the inspection portion on the display after the inspection assistance information is displayed,

### BRIEF DESCRIPTION OF THE DRAWINGS

FIG. 1 is a block diagram showing a co ion of an inspection system according to a first embodiment of the present invention.

FIG. 2 is a perspective view of an endoscope device according to the first embodiment of the present invention.

FIG. 3 is a block diagram showing a configuration of the endoscope device and a server according to the first embodiment of the present invention.

FIG. 4 is a block diagram showing a configuration of a control unit included in the endoscope device according to the first embodiment of the present invention.

FIG. 5 is a block diagram showing a configuration of a control unit included in the server according to the first embodiment of the present invention.

FIG. 6 is a flow chart showing a procedure of processing executed by the endoscope device according to the first embodiment of the present invention.

FIG. 7 is a flow chart showing a procedure of processing executed by the server according to the first embodiment of the present invention.

FIG. 8 is a diagram showing an example of inspection assistance information displayed on a display unit included in the endoscope device according to the first embodiment of the present invention.

FIG. 9 is a diagram showing an example of the inspection assistance information displayed on the display unit included in the endoscope device according to the first embodiment of the present invention.

FIG. 10 is a diagram showing an example of the inspection assistance information displayed on the display unit included in the endoscope device according to the first embodiment of the present invention.

FIG. 11 is a diagram showing an example of the inspection assistance information displayed on the display unit included in the endoscope device according to the first embodiment of the present invention.

FIG. 12 is a diagram showing an example of the inspection assistance information displayed on the display unit eluded in the endoscope device according to the first embodiment of the present invention.

FIG. 13 is a diagram showing an example of the inspection assistance information displayed on the display unit included in the endoscope device according to the first embodiment of the present invention.

FIG. 14 is a diagram showing an example of the inspection assistance information displayed on the display unit included in the endoscope device according to the first embodiment of the present invention.

FIG. 15 is a flow chart showing a procedure of processing executed by an endoscope device according to a first modified example of the first embodiment of the present invention.

FIG. 16 is a diagram showing an example of an information button displayed on a display unit included in the endoscope device according to the first modified example of the first embodiment of the present invention.

FIG. 17 is a flow chart showing a procedure of processing executed by an endoscope device according to a second modified example of the first embodiment of the present invention.

FIG. 18 is a diagram shoving an example of inspection assistance information displayed on a display unit included in the endoscope device according to the second modified example of the first embodiment of the present invention.

FIG. 19 is a diagram showing an example of a structure of an inspection folder in a third modified example of the first embodiment of the present invention.

FIG. 20 is a flow chart showing a procedure of processing executed by an endoscope device according to the third modified example of the first embodiment of the present invention.

FIG. 21 is a diagram showing an example of a change of inspection folder information displayed on a display unit included in the endoscope device according to the third modified example of the first embodiment of the present invention.

FIG. 22 is a flow chart showing a procedure of processing executed by an endoscope device according to a fourth modified example of the first embodiment of the present invention.

FIG. 23 is a diagram showing an example of an image displayed on a display unit included in the endoscope device according to the fourth modified example of the first embodiment of the present invention.

FIG. 24 is a flow chart showing a procedure of processing executed by an endoscope device according to a fifth modified example of the first embodiment of the present invention.

FIG. 25 is a diagram showing an example of an image displayed on a display unit included in the endoscope device according to the fifth modified example of the first embodiment of the present invention.

FIG. 26 is a flow chart showing a procedure of processing executed by an endoscope device according to a sixth modified example of the first embodiment of present invention.

FIG. 27 is a block diagram showing a configuration of an endoscope device according to a second embodiment of the present invention.

FIG. 28 is a block diagram showing a configuration of control unit included in the endoscope device according to the second embodiment of the present invention.

FIG. 29 is a flow chart showing a procedure of processing executed by the endoscope device according to the second embodiment of the present invention.

### DETAILED DESCRIPTION OF THE INVENTION

Hereinafter, embodiments of the present invention will be described with reference to the drawings. An inspection target in each embodiment of the present invention is an industrial product. The inspection target includes one or more inspection portions. For example, in a case in which the inspection target s an aircraft engine, the inspection portion is a combustion tube, a high-pressure turbine, or the like.

The inspection portion may be the entire inspection target. Hereinafter, an example in which an abnormality is damage will be described, For example, the damage is a crack, a dent, or a chip.

### (First Embodiment)

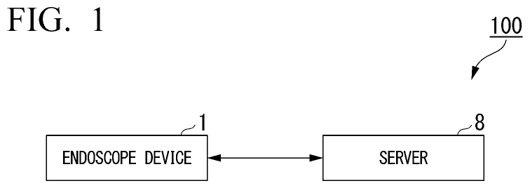

A first embodiment of the present invention will be described. FIG. 1 shows a configuration of an inspection system 100 according to the first embodiment. The inspection system 100 shown in FIG. 1 includes an endoscope device 1 and a server 8. The endoscope device 1 and the server 8 perform communication with each other. For example, the server 8 is a cloud server.

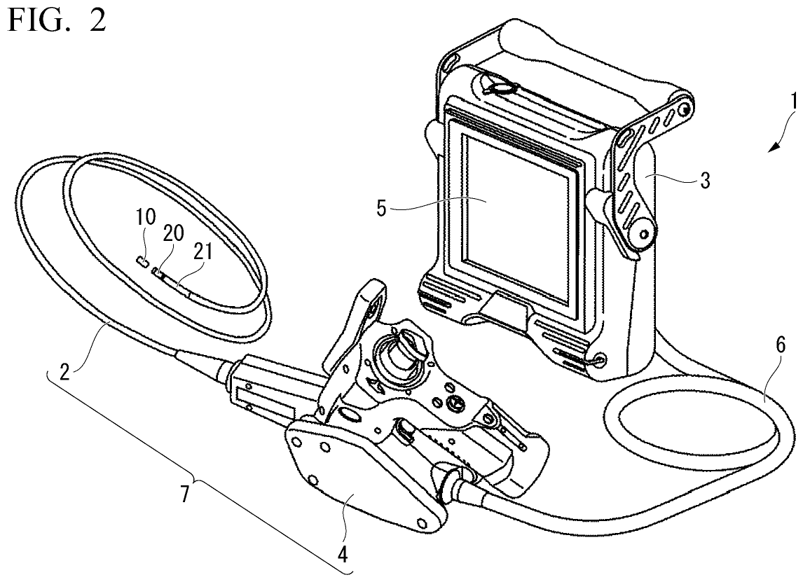

FIG. 2 shows an external appearance of the endoscope device 1. The endoscope device 1 shown in FIG. 2 includes an insertion unit 2, a main body unit 3, an operation unit 4, acid a cable 6.

The insertion unit 2 constitutes an endoscope. The insertion unit 2 is inserted into the inside of an inspection target. A distal end portion 20 is disposed at the distal end of the insertion unit 2. The insertion unit 2 has a long and thin bendable tube shape from the distal end portion 20 to a base end portion. An optical adapter 10 is mounted on the distal end portion 20. A bending portion 21 is disposed on the base end side of the distal end portion 20. The bending portion 21 is capable of bending in a predetermined direction. The base end portion of the insertion unit 2 is connected to the operation unit 4.

The operation unit. 4 is connected to the main body unit 3 by the cable 6. The operation unit 4 includes various buttons including a freeze button and a recording button. In addition, the operation unit 4 includes various joysticks including a joystick used for bending.

A user inputs a still image acquisition instruction to acquire a still image into the endoscope device 1 by operating the freeze button. The user inputs a still-image-recording instruction to record the acquired still image into the endoscope device 1 by operating the recording button. The user inputs a bending instruction to bend the bending portion 21 into the endoscope device 1 by operating the joystick used for bending. The user inputs various kinds of information into the endoscope device 1 by operating a button or the like of the operation unit 4. The operation unit 4 outputs the instruction or the information input by the user to the main body unit 3. The insertion unit 2 and the operation unit 4 constitute a scope 7.

A display unit 5 is disposed on the surface of the main body unit 3. The display unit 5 is a monitor (display) such as a liquid crystal display (LCD). The display unit 5 may be a touch panel, In a case in which the display unit 5 is configured as the touch panel, a user touches the display screen of the display unit 5 by using a part of the body or a tool. For example, the part of the body is the finger.

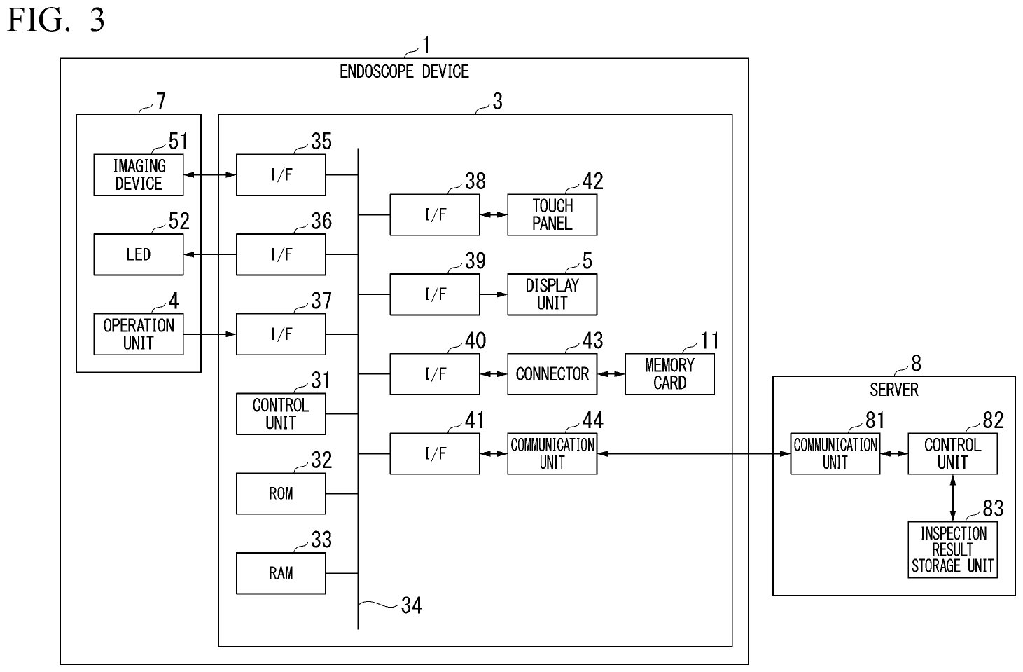

FIG. 3 shows a configuration of the endoscope device 1 and the server A configuration of the scope 7 and the main body unit 3 included in the endoscope device 1 is shown in FIG. 3.

The scope 7 includes the operation unit 4, an imaging device 51, and an LED 52. The imaging device 51 and the LED 52 are disposed in the distal end portion 20. The imaging device 51 is an image sensor such as a CCD sensor or a CMOS sensor. The imaging device 51 sequentially generates two or more images (frames). The two or more images constitute a video. The imaging device 51 outputs each generated image to the main body unit 3. When the still image acquisition instruction is output from the main body unit 3, the imaging device 51 generates a still image and outputs the generated still image to the main body unit 3. The LED 52 generates illumination light. The illumination light generated by the LED 52 is emitted to a subject.

The main body unit 3 includes tine display unit 5, a memory card 11, a control unit 31, a ROM 32, a RAM 33, interfaces (I/Es) 35 to 41, a touch panel 42, a connector 43, and a communication unit 44. The control unit 31, the ROM 32, the RAM 33, and the I/Fs 35 to 41 are connected to each other via a bus 34, Each of the I/Fs 35 to 41 is an interface.

The control unit 31 controls each unit of the endoscope device 1. The control unit 31 is constituted by at least one of a processor and a logic circuit. For example, the processor is at least one of a central processing unit (CPU), a digital signal processor (DSP), and a graphics-processing unit (GPU). For example, the logic circuit is at least one of an application-specific integrated circuit (ASIC) and a field-programmable gate array (FPGA). The control unit nay include one or a plurality of processors. The control unit 31 may include one or a plurality of logic circuits.

A computer of the endoscope device 1 may read a program and execute the read program. The program includes commands defining the operations of the control unit 31. In other words, the functions of the control unit 31 may be realized by software.

The program described above, for example, may be provided by using a “computer readable storage medium” such as a flash memory. The program may be transmitted from the computer storing the program to the endoscope device 1 through a transmission medium or transmission waves in a transmission medium. The “transmission medium” transmitting the program is a medium having a function of transmitting information. The medium having the function of transmitting information includes a network (communication network) such as the Internet and a communication circuit line (communication line) such as a telephone line. The program described above may realize some of the functions described above. In addition, the program described above may be a differential file (differential program). The functions described above may be realized by a combination of a program that has already been recorded in a computer and a differential program.

The ROM 32 stores the above-described program. The RAM 33 temporarily stores information and data processed by the control unit 31. The I/F 35 transmits a driving signal to the imaging device 51 and receives the image output from the imaging device 51. The I/F 36 transmits a driving signal to the LED 52.

The I/F 37 receives the instruction or the information output from the operation unit 4. The I/F 38 outputs a driving signal to the touch panel 42 and receives a signal output from a touch panel 42. The touch panel 42 is built in the screen of the display unit 5. The I/F 39 outputs the image received by the I/F 35 to the display unit 5. display unit disposed outside the endoscope device 1 may be used instead of the display unit 5. The I/F 40 is connected to the memory card 11 via the connector 43. The I/F 40 records the image received by the I/F 35 on the memory card 11 and reads an image from the memory card 11. When the still-image-recording instruction s output from the control unit 31, the I/F 40 records a still image on the memory card 11. The memory card 11 is attachable to and detachable from the connector 43.

The I/F 41 is connected to the communication unit 44. The communication unit 44 is a communicator including a communication circuit. For example, the communication unit 44 is a wireless communicator. The communication unit 44 performs communication with the server 8.

The server 8 includes a communication unit 81, a control unit 82, and an inspection result storage unit 83. The communication unit 81 is a communicator including a communication circuit. For example, the communication unit 81 is a wireless communicator. The communication unit 81 performs communication with the endoscope device 1.

The control unit 82 controls each unit of the server 8. The control unit 82 is constituted by at least one of a processor and a logic circuit. The control unit 82 may include one or a plurality of processors. The control unit 82 may include one or a plurality of logic circuits.

A computer of the server 8 array read a program and execute the read program. The program includes commands defining the operations of the control unit 82. In other words, the functions of the control unit 82 may be realized by software. The program may be realized similarly to that realizing the functions of the control unit 31.

The inspection result storage unit 83 stores inspection result information indicating an inspection result. The inspection result storage unit 83 is a nonvolatile recording medium. For example, the inspection result storage unit 83 is at least one of a static random-access memory (SRAM), an erasable programmable read-only memory (EPROM), an electrically erasable programmable read-only memory (EEPROM), and a flash memory.

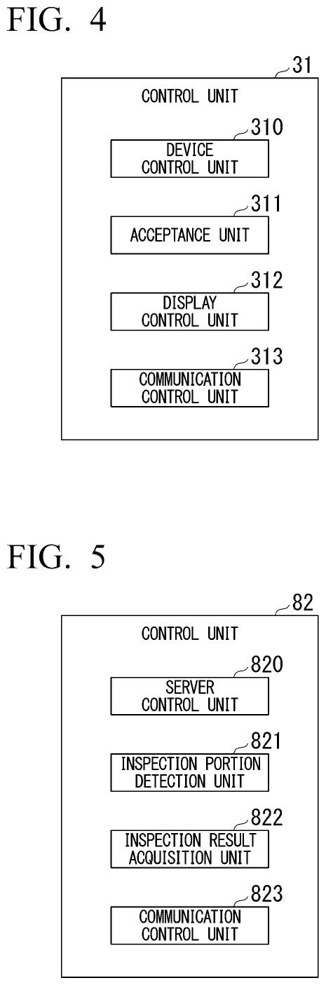

FIG. 4 shows a configuration of t control unit 31. The control unit 31 includes a device control. unit 310, an acceptance unit 311, a display control unit 312, and a communication control unit 313. The device control unit 310 controls each unit of the endoscope device 1.

The acceptance unit 311 accepts the instruction or the information input via the operation unit 4 by a user. When the user operates the freeze button of the operation unit 4, the acceptance unit 311 accepts a still image acquisition instruction and outputs the still image acquisition instruction to the imaging device 51. When the user operates the recording button of the operation unit 4, the acceptance unit 311 accepts a still-image-recording instruction and outputs the still-image recording instruction to the I/F 40.

A still image acquired in an inspection is recorded in an inspection folder in the memory card 11. The inspection folder is prepared for each inspection portion. A user inputs inspection folder information indicating inspection folder into the endoscope device 1 by operating the joystick of the operation unit 4. In this way, the user can select the inspection folder in which the still image is recorded. When the user inputs the inspection folder information, the acceptance unit 311 accepts the inspection folder information. Since the inspection folder and the inspection portion are associated with each other, the inspection folder information indicates the inspection portion.

The display control unit 312 outputs the image received by the I/F 35 to the display unit 5 via the I/F 39. In this way, the display control unit 312 displays the image on the display unit 5.

As described later, inspection result information is received from the server 8. The inspection result information indicates a previous inspection result of the inspection portion corresponding to the inspection folder information. The display control unit 312 outputs the inspection result information as inspection assistance information to the display unit 5 via the I/F 39. Alternatively, the display control unit 312 outputs inspection assistance information generated on the basis of the inspection result information to the display unit 5 via the I/F 39. In this way, the display control unit 312 displays the inspection assistance information on the display unit 5. The inspection assistance information notifies a user of information related to an abnormality of an inspection portion detected in a previous inspection. For example, the inspection assistance information is an image of the inspection portion. The inspection assistance information may include information related to a state or the like of an abnormality found in a previous inspection.

For example, the image of the inspection portion is a two-dimensional image (2D image) generated by the imaging device 51. The image of the inspection portion may be a three-dimensional image (3D image) generated by using the 2D image.

The communication control unit 313 controls the communication unit 44 via the I/F 41 and performs communication with the server 8. The communication control unit 313 transmits the inspection folder information to the server 8 by using the communication unit 44. In addition, the communication control unit 313 receives the inspection result information from the server 8 by using the communication unit 44. The communication control unit 313 may transmit an image such as a still image to the server 8 by using the communication unit 44.

FIG. 5 shows a configuration of the control unit 82. The control unit 82 includes a server control unit 820, an inspection portion detection unit 821, an inspection result acquisition unit 822, and a communication control unit 823.

The server control unit 820 controls each unit of the server 8. The inspection portion detection unit 821 detects an inspection portion corresponding to the inspection folder indicated by the inspection folder information. The inspection result acquisition unit 822 acquires the inspection result information of the inspection portion detected by the inspection portion detection unit 821 from the inspection result storage unit 83.

The communication control unit 823 controls the communication unit 81 and performs communication with the endoscope device 1. The communication control unit 823 receives the inspection folder information from the endoscope device 1 by using the communication unit 81. In addition, the communication control unit 823 transmits the inspection result information to the endoscope device 1 by using the communication unit 81. The communication control unit 823 may receive an image such as a still image from the endoscope device 1 by using the communication unit 81. The received image may be recorded on the inspection result storage unit 83.

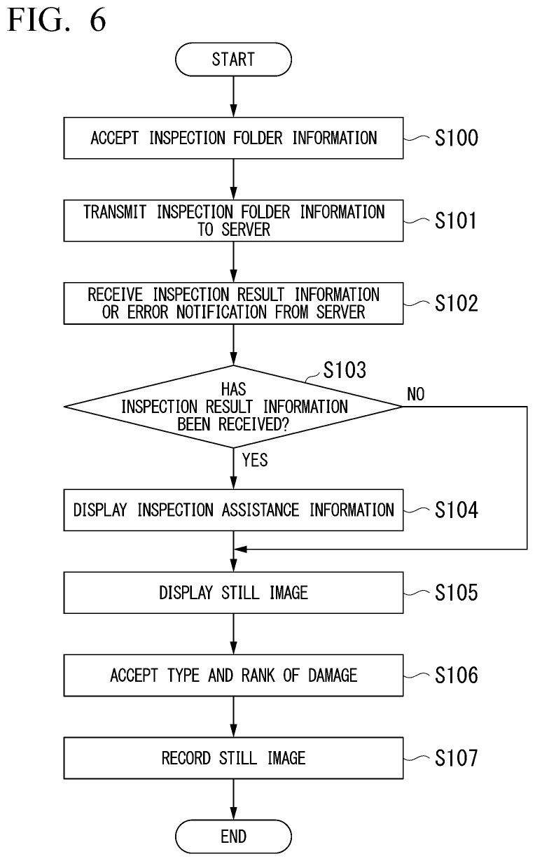

Processing executed by the endoscope device 1 will be described by using FIG. 6. FIG. 6 shows a procedure of the processing executed by the endoscope device 1.

The imaging device 51 sequentially generates images (frames). The display control unit 312 outputs the images generated by the it raging device 51 to the display unit 5 via the I/F 39. The display unit 5 sequentially displays the images, thus displaying live images of a subject. Processing of generating and displaying images of the subject is executed in parallel with the processing shown in FIG. 6.

Before an inspection is started, or when the inspection is started, a user inputs inspection folder information into the endoscope device 1 by using the joystick of the operation unit 4. The acceptance unit 311 accepts the inspection folder information (Step S100).

After Step S100, the communication control unit 313 outputs the inspection folder information to the communication unit 44 via the I/F 41 and transmits the inspection folder information to the server 8 (Step S101).

After Step S101, the communication control unit 313 receives inspection result information or an error notification from the server 8 by using the communication unit 44 (Step S102).

When the inspection result information of the inspection portion corresponding to the inspection folder information is recorded on the inspection result storage unit 83 of the server 8, the server 8 transmits the inspection result information to the endoscope device 1. The inspection result information is received in Step S102. When the inspection result information of the inspection portion corresponding to the inspection folder information is not recorded on the inspection result storage unit 83 of the server 8. the server 8 transmits the error notification to the endoscope device 1. In this case, the error notification is received in Step S102.

After Step S102, the device control unit 310 determines whether the inspection result information has been received (Step S103). In a case in which the error notification is received in Step S102, the device control unit 310 determines that the inspection result information has not been received. In a case in which the inspection result information is received in Step S102, the device control unit 310 determines that the inspection result information has been received.

When the device control unit 310 determines that the inspection result information has not been received in Step S103, Step S105 described later is executed. When the device control unit 310 determines that the inspection result information has been received in Step S103, the display control unit 312 outputs the inspection result information as inspection assistance information to the display unit 5 via the I/F 39. Alternatively, the display control unit 312 generates inspection assistance information on the basis of the inspection result information and outputs the inspection assistance information to the display unit 5 via the I/F 39. In this way, the display control unit 312 displays the inspection assistance information on the display unit 5 (Step S104).

The inspection assistance information is displayed on the display unit 5 before an inspection of an inspection portion scheduled to be inspected is performed. A user can recognize the type and the state of damage previously detected in the inspection portion by checking the inspection assistance information. The user can perform an inspection in a state in which he/she recognizes the type and the state of damage. Therefore, a possibility that the user overlooks an abnormality or mistakes evaluation of the abnormality is reduced.

Thereafter, the user checks the live image displayed on the display unit 5 and moves the distal end portion 20 of the insertion unit 2 to the inspection portion. The user operates the freeze button of the operation unit 4 in order to acquire a still image. At this time, the acceptance unit 311 accepts a still image acquisition instruction and outputs the still image acquisition instruction to the imaging device 51. The imaging device 51 generates a still image and outputs the generated still image to the main body unit 3. The I/F 35 receives the still image and outputs the still image to the control unit 31. The display control unit 312 outputs the still image to the display unit 5 via the I/F 39. The display unit 5 displays the still image (Step S105).

After the still image is displayed, the user inputs a type and a rank of damage into the endoscope device 1 by operating the operation unit 4. The acceptance unit 311 accepts the type and the rank of damage (Step S106).

For example, the user selects the type of damage from a list registered in advance. The rank of damage indicates a state (severity) of the damage. For example, the rank of damage is any one of “no problem (Accept),” “replacement required (Reject),” “repair required (Repair),” and “re-inspection required (Re-Inspect).” As the rank of damage, “no mark (No Mark)” may be designated.

After Step S106, the device control unit 310 records the still image on the memory card 11 via the I/F 40 (Step S107). At this time, the still image is recorded in the inspection folder indicated by the inspection folder information. When Step S107 is executed, the processing shown in FIG. 6 is completed.

The file name of the still image recorded on the memory card 11 includes a folder name, a sequence number, and a file mark. For example, the file format of the still image is JPEG. The type of damage is included in EXIF information.

The folder name is a name of a folder in which the still image is recorded. The sequence number is a number following the last number included in the file name of the still image recorded in the folder. The file mark is a character indicating the rank of damage. For example, the file mark corresponding to “no problem (Accept)” is a character “A.” The file mark corresponding to “replacement required (Reject)” is a character “B.” The file mark corresponding to “repair required (Repair)” is a character “C.” The file mark corresponding to “re-inspection required (fie-Inspect)” is a character “D.” The file mark corresponding to “no mark (No Mark)” is a character “X.” In a case in which “no mark (No Mark)” is designated as a rank of damage, a file name not including a file mark may be used.

For example, the folder name is “HPC_STAGE1_ZONE” A still image including a number 00005 in the file name is recorded in a folder having such a folder name. For example, the file mark “A” is selected. In this case, the file name of the still image is “HPC_STAGE1_ZONE_00005_A.JPG.”

After the processing shown in FIG. 6 is completed, the device control unit 310 generates inspection result information indicating an inspection result this time. For example, the inspection result information includes the still image displayed on the display unit 5 in Step S105. The inspection result information a may include information indicating the type and the rank of damage. The communication control unit 313 outputs the inspection result information to the communication unit 44 via the I/F 41 and transmits the inspection result information to the server 8.

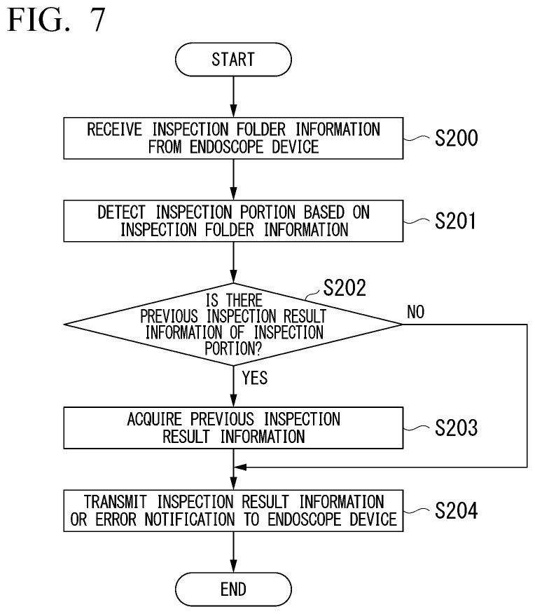

Processing executed by the server 8 will be described by using FIG. 7. FIG. 7 shows a procedure of the processing executed by the server 8.

The communication control unit 823 receives inspection folder information from the endoscope device 1 by using the communication unit 81 (Step S200).

After Step S200, the inspection portion detection unit 821 detects an inspection portion on the basis of the inspection folder information received in Step S200 (Step S201). The inspection folder is prepared for each inspection portion and is associated with the inspection portion. A relationship between the inspection folder and the inspection portion is set in advance. For example, the inspection result storage unit 83 stores information indicating the relationship. The inspection portion detection unit 821 detects the inspection portion associated with the inspection folder indicated by the inspection folder information in Step S201.

After Step S201, the inspection result acquisition unit 822 searches the inspection result storage unit 83 for the inspection result information of the inspection portion detected in Step S201. The inspection result acquisition unit 822 determines whether there is previous inspection result information of the inspection portion on the basis of the result of the search (Step S202).

In a case in which the previous inspection result information of the inspection portion detected in Step S201 is stored on the inspection result storage unit 83, the inspection result acquisition unit 822 determines that there is the previous inspection result information of the inspection portion. In a case in which the previous inspection result information of the inspection portion detected in. Step S201 is not stored on the inspection result storage unit 83, the inspection result acquisition unit 822 determines that there is no previous inspection result information of the inspection portion.

When the inspection result acquisition unit 822 determines that there is the previous inspection result information of the inspection portion in Step S202, the inspection result acquisition unit 822 acquires the inspection result information from the inspection result storage unit 83 (Step S203).

After Step S203, the communication control unit 823 transmits the inspection result information acquired in Step S203 to the endoscope device 1 by using the communication unit 81. When the inspection result acquisition unit 822 determines that there is no previous inspection result information of the inspection portion in Step S202, the communication control unit 823 transmits an error notification to the endoscope device 1 by using the communication unit 81 (Step S204). When Step S204 is executed, the processing shown in FIG. 7 is completed.

There is a possibility that the inspection portion is changed. For example, the distal end portion 20 of the insertion unit 2 is disposed at a first position, and an inspection of a first inspection portion is performed. After the inspection of the first inspection portion is performed, the distal end portion 20 of the insertion unit 2 is disposed at a second position and an inspection of a second inspection portion is performed. The second position is different from the first position, and the second inspection portion is different from the first inspection portion.

An image of e second inspection portion may be recorded on the memory card 11 in advance. Alternatively, the communication control unit 313 may receive the image of the second inspection portion from the server 8 by using the communication unit 44. The device control unit 310 may execute image processing of comparing the image generated by the imaging device 51 with the image of the second inspection portion, When the image generated by the imaging device 51 is similar to the image of the second inspection portion, the device control 310 may determine that the second inspection portion is seen in the image. In this way, the device control unit 310 can detect that the distal end portion 20 of the insertion unit 2 is disposed at the second position. At this time, the display control unit 312 may display the inspection assistance information related to the second inspection portion on the display unit 5.

Another example in which the device control unit 310 detects that the distal end portion 20 of the insertion unit 2 is disposed at the second position will be described.

The distal end portion 20 of the insertion unit 2 may include a gravity sensor. The gravity sensor determines the direction of gravity applied to the distal end portion 20. Since the gravity sensor is fixed in the distal end portion 20, the relative direction of the gravity sensor to the distal end portion 20 does not change. When the direction of the distal end portion 20 for the direction of the gravity changes, the direction of the gravity determined by the gravity sensor changes. Therefore, the device control unit 310 can determine the proving direction of the distal end portion 20.

Two rollers may be disposed so as to locate the base end portion of the insertion unit herebetween, and a rotary encoder may be disposed. The two rollers are in contact with the insertion unit 2. The two rollers rotate as the insertion unit 2 moves.

The rotary encoder determines the amount of rotation of at least one of the two rollers, thus determining the length (insertion length) of a portion of the insertion unit 2 inserted into an inspection target. The rotary encoder outputs information indicating determined insertion length to the device control unit 310.

Path information may be recorded on the memory card 11 in advance. The path information indicates a path to each inspection portion scheduled to be inspected. The communication control unit 313 pray receive the path information from the server 8 by using the con it cation unit 44.

The device control unit 310 calculates the position of the distal end portion 20 on the basis of the moving direction of the distal end portion 20 and the insertion length of the insertion unit 2. When the position of the distal end portion 20 becomes the position of the second inspection portion indicated by the path information, the device control unit 310 determines that the distal end portion 20 is disposed at the second position. At this the display control unit 312 may display the inspection assistance information related to the second inspection portion on the display unit 5.

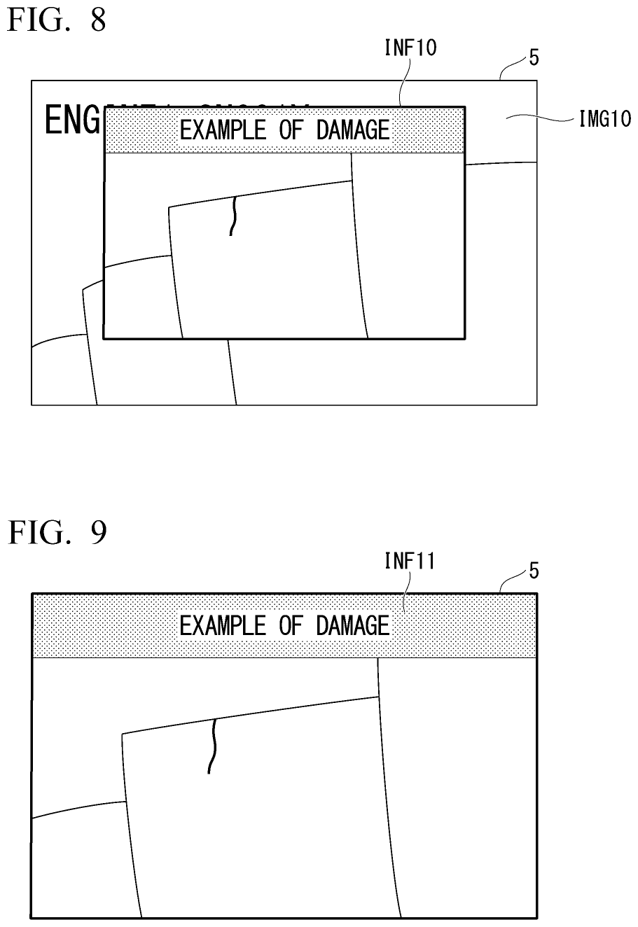

Examples of the inspection assistance information will be described by using FIGS. 8 to 14. FIGS. 8 to 14 show the examples of the inspection assistance information displayed on the display unit 5 in Step S104.

FIG. 8 shows inspection assistance information INF10 and a live image IMG10 displayed on the display unit 5. The live IMG10 is displayed on the display unit 5, and the inspection assistance information INF10 is displayed on the live image IMG10.

The inspection assistance information INF10 is an image of a damaged inspection portion. The inspection assistance information INF10 overlaps the live image IMG10 and conceals part of the live image IMG10.

The inspection assistance information INF10 may be a reference image. The reference image is an image of a damage model. For example, CAD data of the inspection portion are processed, and damage is added at the inspection portion. By converting the processed data into a 2D image or a 3D image, the reference image is generated.

FIG. 9 shows inspection assistance information INF11 displayed on the display unit 5. The inspection assistance information INF11 is an image of a damaged. inspection portion. The inspection assistance information INF11 is displayed on the entire screen of the display unit 5.

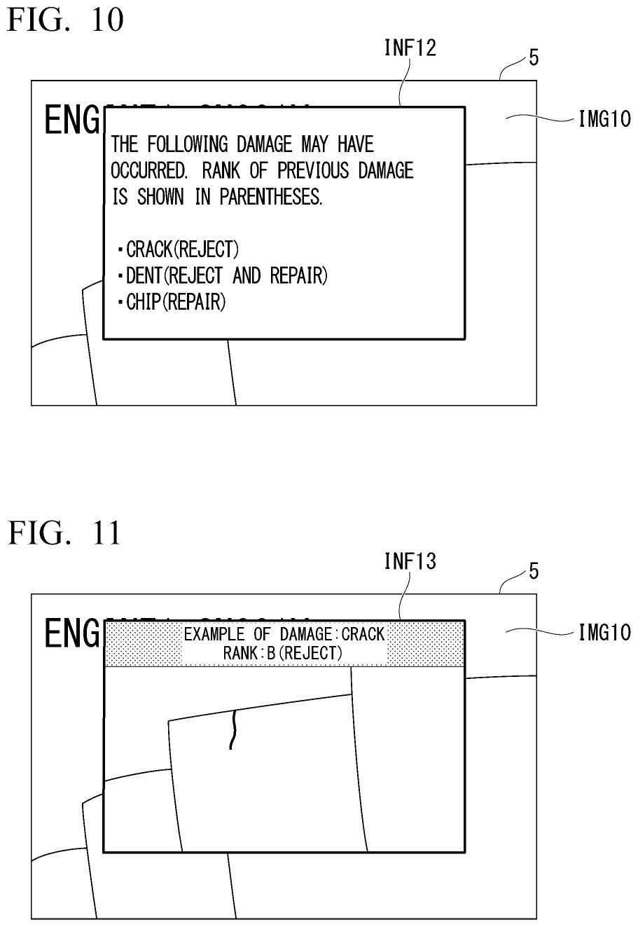

FIG. 10 shows inspection assistance information INF12 and a live image IMG10 displayed on the display unit 5. The live image IMG10 is displayed on the display unit 5, and the inspection assistance information INF12 is displayed on the live image IMG10. The inspection assistance information INF12 includes characters indicating the type and the rank of damage. The inspection assistance information INF12 overlaps the live image IMG10 and conceals part of the live image IMG10. The inspection assistance information INF12 may be displayed on the entire screen of the display unit 5.

FIG. 11 shows inspection assistance information INF13 and a live image IMG10 displayed on the display unit 5. The live image IMG10 is displayed on the display unit 5, and the inspection assistance information INF13 is displayed on the live image IMG10. The inspection assistance information INF13 includes an image of a damaged inspection portion and characters indicating the type and the rank of the damage. The inspection assistance information INF13 overlaps the live image IMG10 and conceals part of the live image IMG10. The inspection assistance information INF13 may be displayed on the entire screen of the display unit 5.

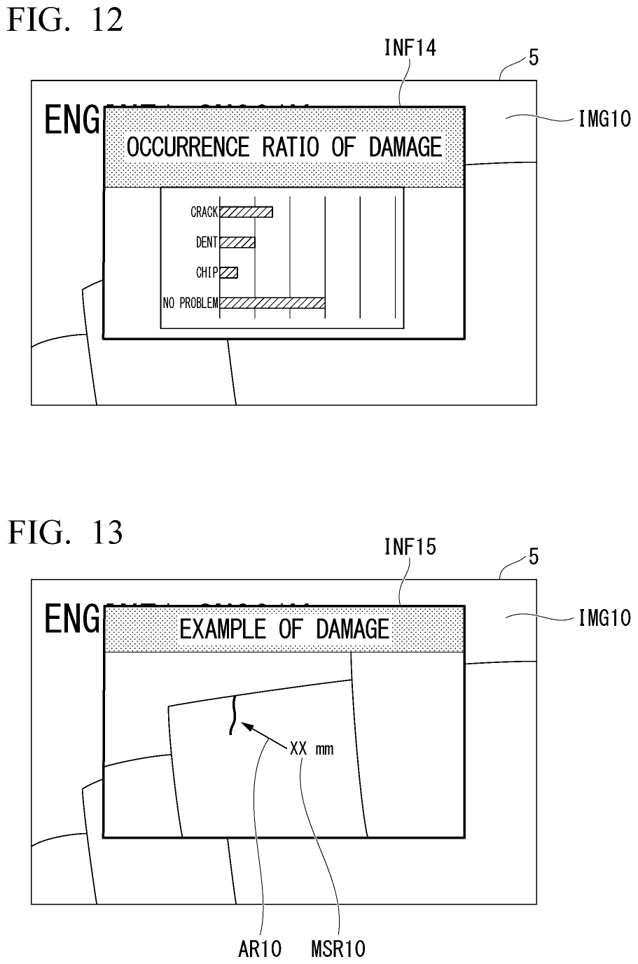

FIG. 12 shows inspection assistance information INF14 and a live image IMG10 displayed on the display unit 5. The live image IMG10 is displayed on the display unit 5, and the inspection assistance information INF14 is displayed on the live image IMG10. The inspection assistance information INF14 indicates an occurrence ratio for each type of damage previously detected in an inspection portion. The occurrence ratio indicates a proportion of the number of pieces of damage for each type to the number of all pieces of damage previously detected in the inspection portion. The inspection assistance information INF14 may indicate an occurrence number for each type of damage previously detected in the inspection portion. The occurrence number indicates the number of pieces of damage for each type previously detected in the inspection portion. The inspection assistance information INF14 overlaps the live image IMG10 and conceals part of the live image IMG10. The inspection assistance information INF14 may be displayed on the entire screen of the display unit 5.

When the inspection result information transmitted from the endoscope device 1 is saved on the inspection result storage unit 83 of the server 8, the server control unit 820 may calculate an occurrence ratio for each type of damage or an occurrence number for each type of damage on the basis of the inspection result information stored on the inspection result storage unit 83. The server control unit 820 may save the calculated occurrence ratio or occurrence number on the inspection result storage unit 83. The inspection result acquisition unit 822 may acquire the occurrence ratio or the occurrence number as the inspection result information from the inspection result storage unit 83 in Step S203.

When the inspection result acquisition unit 822 acquires the inspection result information from the inspection result storage unit 83 in Step S203, the server control unit 820 may calculate an occurrence ratio for each type of damage or an occurrence number for each type of damage on the basis of the inspection result information. The communication control unit 823 may transmit the occurrence ratio or the occurrence number as the inspection result information to the endoscope device 1 in Step S204.

FIG. 13 shows inspection assistance information INF15 and a live image IMG10 displayed on the display unit 5. The live image IMG10 is displayed on the display unit 5, and the inspection assistance information INFI5 is displayed. on the live image IMG10. The inspection assistance information INF15 includes an image of a damaged inspection portion, information indicating a position of a measurement target, and information indicating a measurement result. The inspection assistance information INF15 overlaps the live image IMG10 and conceals part of the live image IMG10. The inspection assistance information INF15 may be displayed on the entire screen of the display unit 5.

The inspection assistance information INF15 includes an arrow AR10 and a measurement value MSR10. The arrow AR10 indicates damage (measurement target) previously detected in the inspection portion. The measurement value MSR10 indicates a measurement result of the size (length or depth) of the damage. When a still image is recorded on the memory card 11, the arrow AR10 and the measurement value MSR10 are attached to the inspection assistance information INF15 by a user. Alternatively, after the still image is recorded on the memory card 11, the arrow AR10 and the measurement value MSR10 are attached to the inspection assistance information INF15 at any timing by the user.

There is a case in which the types or the ranks are the same as or similar to each other between two or a more pieces of damage. Since the measurement value of damage is displayed as the inspection assistance information, a user can determine the degree of importance of the two or more pieces of damage by referring to the measurement value.

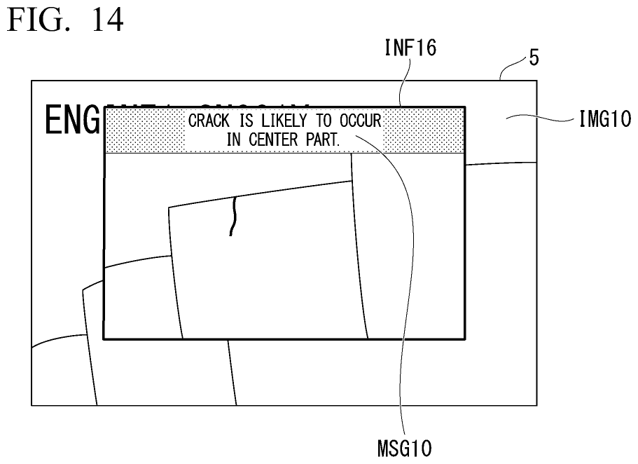

FIG. 14 shows inspection assistance information INF16 and a live image IMG10 displayed on the display unit 5. The live image IMG10 is displayed on the display unit 5, and the inspection assistance information INF16 is displayed on the live image IMG10. The inspection assistance information INF16 includes an in image of a damaged inspection portion and a message related to the damage. The inspection assistance information INF16 overlaps the live image IMG10 and conceals part of the live image IMG10. The inspection assistance information INF16 may be displayed on the entire screen of the display unit 5.

The inspection assistance information INF16 includes a message MSG10. The message MSG10 includes a character string indicating damage of which the occurrence ratio or the occurrence number is the greatest. In other words, the message MSG10 includes a character string indicating the occurrence tendency of damage. In the example shown in FIG. 14, the occurrence ratio car occurrence number of a crack is the greatest. In addition, the message MSG10 includes a character string indicating the position of the damage. In the example shown in FIG. 14, the position of the damage is a center part of the inspection portion.

The message MSG10 relates to a crack in the center part of the inspection portion. The message displayed on the display unit 5 may indicate a dent in the distal end portion of the inspection portion, a chip in the distal end portion of the inspection portion, or the like. In a case in which the inspection portion includes two or more similar components, a message may be displayed for each component. For example, in a case in which the inspection portion includes two or more blades in a turbine, the message may indicate a type or a position of damage in a specific blade.

There is a case in which the types, the occurrence ratios, or the occurrence numbers are the same as or similar to each other between two or more pieces of damage. Since the message indicating the type, the position, and the occurrence tendency of damage is displayed, a user can predict a type and a position of damage to which attention should be paid. Therefore, a possibility that the user overlooks the damage is reduced.

The display control unit 312 may set a transmittance (transparency) of the inspection assistance information displayed on all live image. For example, the transmittance ranges from 0% to 100%. When the transmittance is 0%, the live image is not seen through the inspection assistance information in a region (overlapping region) in which the inspection assistance information overlaps the live image. A user can visually recognize the inspection assistance information but cannot visually recognize the live image in the overlapping region. When the transmittance is greater than 0% and less than 100%, the live image is seen through the inspection assistance information in the overlapping region. The user can visually recognize the inspection assistance information and the live image in the overlapping region. When the transmittance is 100%, the inspection assistance information is transparent. The user can visually recognize the live image in the overlapping region but cannot visually recognize the inspection assistance information.

For example, the display control unit 312 sets the transmittance to 0% in Step S104. The display control unit 312 may set the transmittance to a greater value than 0% in Step S104.

After the inspection assistance information is displayed on the live image, a user may input an instruction to change the transmittance into the endoscope device 1 by operating the operation unit 4 or the touch panel 42. For example, the user may input an instruction to increase the transmittance into the endoscope device 1. The acceptance unit 311 may accept the instruction. The display control unit 312 may change the transmittance on the basis of the instruction. Since the transmittance is variable, the visibility of the live image by the user can be improved.

The inspection assistance information does not need to be displayed along with a live image. The display control unit 312 may display the live image on the display unit 5 after displaying the inspection assistance information on the display unit 5.

After the inspection assistance information is displayed on the display unit 5, the display control unit 312 may hide the inspection assistance information in accordance with movement of the insertion unit 2 or an operation for bending. Alternatively, the display control unit 312 may reduce the size of a display region in which the inspection assistance information is displayed in accordance with the movement of the insertion unit 2 or the operation for bending. The size of the display region after this control is executed is less than that of the display region before this control is executed. For example, when the insertion unit 2 moves in a state in which the inspection assistance information is displayed on the display unit 5, the display control unit 312 may hide the inspection assistance information or may reduce the size of the display region. Alternatively, when a user inputs a bending instruction into the endoscope device 1 in a state in which the inspection assistance information is displayed on the display unit 5, the display control unit 312 may hide the inspection assistance information or may reduce the size of the display region.

The display control unit 312 may change the transmittance of the inspection assistance information in accordance with movement of the insertion unit 2 or an operation for bending. For example, when the insertion unit 2 moves in a state in which the inspection assistance information is displayed on the display unit 5, the display control unit 312 may increase the transmittance. Alternatively, when a user inputs a bending instruction into the endoscope device 1 in a state in which the inspection assistance information is displayed on the display unit 5, the display control unit 312 may increase the transmittance. In these cases, the display control unit 312 may set the transmittance to 100%.

In a case in which the distal end portion 20 of the insertion unit 2 includes an acceleration sensor, the device control unit 310 may determine movement of the insertion unit 2 on the basis of an acceleration determined by the acceleration sensor. The device control unit 310 may calculate the amount of movement of a subject in an image generated by the imaging device 51 and may determine movement of the insertion unit 2 on the basis of the amount of the movement.

Another example of processing in Step S203 will be described. A model number is given to an inspection target in accordance with the type of the inspection target. In addition, a serial number is given to an individual of the inspection target. When the model number of an inspection target A is the same as that of an inspection target B, the inspection target A and the inspection target B are inspection targets of the same type. When the model number of an inspection target A is the same as that of an inspection target B and the serial number of the inspection target A is different from that of the inspection target B, the inspection target A and the inspection target B are inspection targets of the same type and are different individuals.

For example, the inspection result acquisition unit 822 acquires the inspection result information of an inspection target A1 including an inspection portion detected in Step S201 from the inspection result storage unit 83. There is a case in which previous inspection result information of the inspection target A1 does not exist. In such a case, the inspection result acquisition unit 822 may acquire the inspection result information of an inspection target A2 from the inspection result storage unit 83. The model number of the inspection target A1 is the same as that of the inspection target A2. The serial number of the inspection target A1 is different from that of the inspection target A2. In other words, the inspection target A1 and the inspection target A2 are inspection targets of the same type and are different individuals. In a case in which the previous inspection result information of the inspection target A1 does not exist, the inspection result acquisition unit 822 may acquire a reference image of the inspection target A1 from the inspection result storage unit 83.

In a case in which a 3D image is displayed as the inspection assistance information, the 3D image array be rotated in accordance with an operation performed by a user. Specifically, the user may input an instruction to rotate the 3D image into the endoscope device 1 by operating the operation unit 4 or the touch panel 42. The acceptance unit 311 may accept the instruction. The display control unit 312 may change the direction of an inspection portion in the 3D image by rotating the 3D image on the basis of the instruction.

In a case in which a 2D image is displayed as the inspection assistance information, a user can check an inspection portion seen only in one direction. In a case in which a 3D image is displayed as the inspection assistance information, the 3D image includes stereoscopic information (depth or the like). Therefore, the user can check an inspection portion seen in various directions by rotating the 3D image. The user can check an abnormality previously detected in the inspection portion from various viewpoints, and a possibility that the user overlooks the abnormality is reduced.

The control unit 31 may generate a 3D image by using a known technique. Hereinafter, two methods of generating a 3D image will be described.

First, a method of generating a 3D image by using one or more stereo images will be described. The stereo image includes a 2D image (first 2D image) of a subject seen from a first viewpoint and a 2D image (second 2D image) of the subject seen from a second viewpoint different from the first viewpoint. A stereo optical system having two different visual fields is mounted as the optical adaptor 10 on the distal end portion 20 of the insertion unit 2.

The stereo optical system includes a first optical system and a second optical system. The endoscope device 1 may switch between a first state and a second state. In the first state, only light passing through the first optical system is incident on the imaging device 51. In the second state, only light passing through the second optical system is incident on the imaging device 51. The imaging device 51 generates a first 2D image in the first state and generates a second 2D image in the second state. A combination of the first 2D image and the second 2D image constitutes a stereo image. The control unit 31 generates a 3D image by using one or more first 2D images and one or m second 2D images.

Next, another method of generating a 3D image will be described. A single-eye optical system having a single visual field is mounted as the optical adaptor 10 on the distal end portion 20 of the insertion unit 2. Two or more images are acquired when the single-eye optical system is mounted on the distal end portion 20 of the insertion unit 2. The control unit 31 generates a 3D image by applying a technique called structure from motion (SfM) to the two or more images.

An inspection assistance method according to each aspect of the present invention includes an acceptance step, an acquisition step, an inspection assistance step, and an image display step. The acceptance unit 311 accepts inspection portion information (inspection folder information) related to an inspection portion included in at least part of an inspection target (acceptance step (Step S100)). The inspection result acquisition unit 822 connects to a storage medium (inspection result storage unit 83) storing a result of a previous inspection and acquires the result of the inspection of the inspection portion from the storage medium (acquisition step (Step S203)). The display control unit 312 displays inspection assistance information on the display unit 5 on the basis of the result acquired in the acquisition step (inspection assistance step (Step S104)). The inspection assistance information notifies a user of information related to an abnormality of the inspection portion detected in the previous inspection. After the inspection assistance information is displayed, the display control unit 312 displays an image of the inspection portion on the display unit 5 (image display step (Step S105)). The image may be a still image or a live image generated by the imaging device 51 after the inspection assistance information is displayed.

An inspection assistance system according to each aspect of the present invention includes the endoscope device 1 (inspection assistance device) and the server 8. The endoscope device 1 includes the acceptance unit 311, the communication unit 44 (first communication unit), and the display control unit 312 (an inspection assistance unit and an image display unit). The acceptance unit 311 accepts inspection portion information (inspection folder information) related to an inspection portion included in at least part of an inspection target. The communication unit 44 transmits the inspection portion information to the server 8 and receives a result of a previous inspection of the inspection portion from the server 8. The display control unit 312 displays inspection assistance information on the display unit 5 on the basis of the result received by the communication unit 44. The inspection assistance information notifies a user of information related to an abnormality of the inspection portion detected in the previous inspection. After the inspection assistance information is displayed, the display control unit 312 displays an image of the inspection portion on the display unit 5. The server 8 includes the inspection result acquisition unit 822 and the communication unit 81 (second communication unit). The inspection result acquisition unit 822 connects to a storage medium (inspection result storage unit 83) storing the result of the previous inspection and acquires the result of the inspection of the inspection portion corresponding to the inspection portion information from the storage medium. The communication unit 81 receives the inspection portion information from the endoscope device 1 and transmits the result acquired by the inspection result acquisition unit 822 to the endoscope device 1.

Each aspect of the present may include the following modified example. The inspection portion information (inspection folder information) indicates a folder of the storage medium (inspection result storage unit 83) in which the result of the previous inspection is saved. The folder is prepared for each inspection portion and is associated with the inspection portion. The inspection result acquisition unit 822 acquires the result of the inspection of the inspection portion from the folder indicated by the inspection portion information in the acquisition step (Step S203).

Each aspect of the present invention may include the following modified example. The inspection assistance information (INF10, INF11, and INF13) includes an image of an abnormality detected in the previous inspection. For example, the image is acquired when the inspection portion s inspected. The image may be a reference image. The image of the abnormality is a 2D image or a 3D image. The reference image is a 2D image or a 3D image.

Each aspect of the present invention may include the following modified example. The inspection assistance information indicates the number of abnormalities for each type of the abnormalities.

Each aspect of the present invention may include the following modified example. The inspection assistance information (INF14) indicates a proportion of the number of abnormalities previously detected in the inspection portion for each type of the abnormalities to the number of all abnormalities previously detected in the inspection portion.

Each aspect of the present invention may include the following modified example. The inspection result acquisition unit 822 acquires the result of the inspection previously performed for the same object as the inspection target or for an object of the same type as that of the inspection target in the acquisition step (Step S203).

Each aspect of the present invention may include the following modified example. The inspection result acquisition wait 822 searches for the result of the inspection of the inspection portion in the same object as the inspection target in the acquisition step (Step S203). In a case in which the result of the inspection portion is not acquired, the inspection result acquisition unit 822 acquires the result of the inspection of the inspection portion in an object of the same type as that of the inspection target in the acquisition step (Step S203).

Each aspect of the present invention may include the following modified example. The display control unit 312 displays one of two or more sequentially generated images (live images) of the inspection portion on the display unit 5 in the inspection assistance step (Step S104).

Each aspect of the present invention may include the following modified example. Alter the inspection assistance information is displayed, the display control unit 312 hides the inspection assistance information or reduces the size of a region of the inspection assistance information displayed on the display unit 5 when movement of the insertion unit 2 inserted into the inspection target is detected.

Each aspect of the present invention lay include the following modified example. The display control unit 312 displays the inspection assistance information on an image of the inspection portion in the inspection assistance step (Step S104). After the inspection assistance information is displayed, the display control unit 312 changes the transmittance of the inspection assistance information when movement of the insertion unit 2 inserted into the inspection target is detected.

In the first embodiment. the endoscope device 1 displays the inspection assistance information on the display unit 5. Therefore, the endoscope device 1 can reduce a possibility that a user overlooks an abnormality or mistakes evaluation of the abnormality.

### (First Modified Example of First Embodiment)

A first modified example of the first embodiment of the present invention will be described. The display control unit 312 displays an information button on the screen of the display unit 5. When the information button is pressed, the display control unit 312 displays the inspection assistance information on the display unit 5.

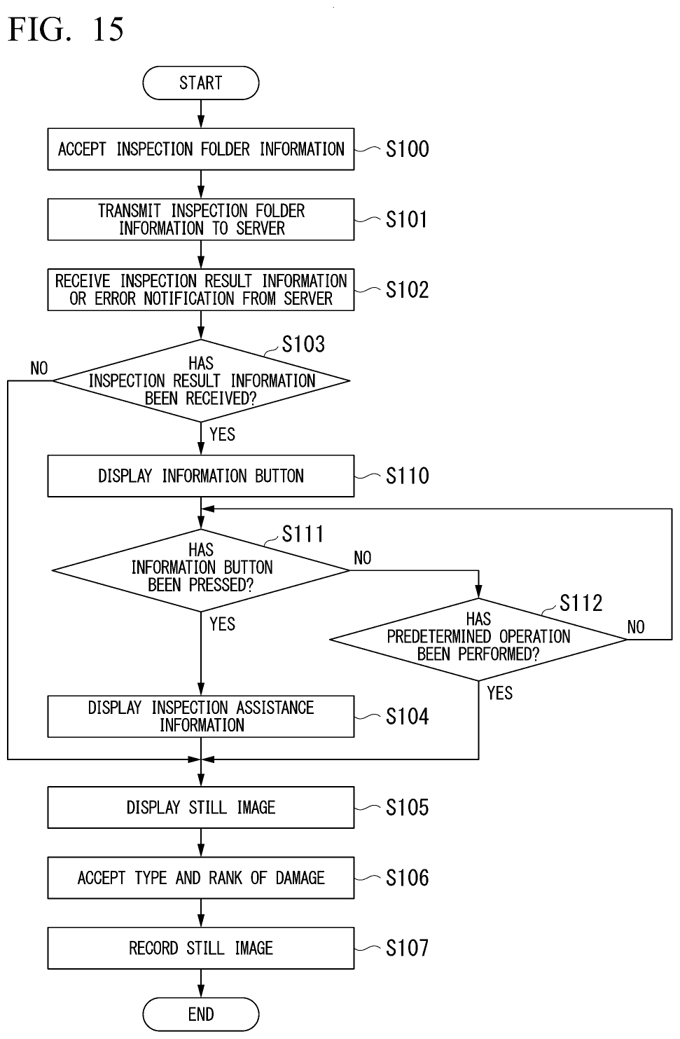

Processing executed by the endoscope device 1 will be described by using FIG. 15. FIG. 15 shows a procedure of the processing executed by the endoscope device 1. The same processing as that shown in FIG. 6 rill not be described.

When the device control unit 310 determines that the inspection result information has been received in Step S103, the display control unit 312 outputs an image of the information button to the display unit 5 via the I/F 39. In this way, the display control unit 312 displays the information button on the display unit 5 (Step S110).

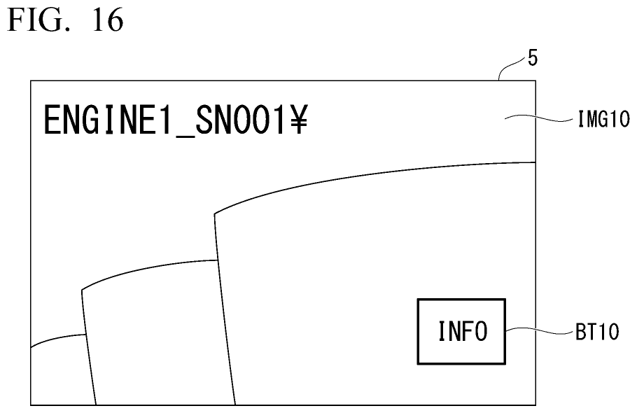

FIG. 16 shows an information button BT10 and a live image IMG10 displayed on the display unit 5 in Step S110. The live image IMG10 is displayed on the display unit 5, and the information button BT10 is displayed on the live image IMG10.

A user inputs an instruction press the information button BT10 into the endoscope device 1 by operating the operation unit 4. Alternatively, the user inputs instruction to press the information button BT10 into the endoscope device 1 by operating the touch panel 42. The instruction output from the operation unit 4 is input into the control unit 31 via the I/F 37. Alternatively, the instruction output from the touch panel 42 is input into the control unit 31 via the I/F 38.

After Step S110, the device control unit 310 determines whether the information button BT10 has been pressed (Step S111l). When the instruction to press the information button BT10 is not output from the operation unit 4 or the touch panel 42, the device control unit 310 determines that the information button BT10 has not been pressed. When the instruction to press the information button BT10 is output from the operation unit 4 or the touch panel 42, the device control unit 310 determines that the information button BT10 has been pressed.

When the device control unit 310 determines that the information button BT10 has been pressed in Step S111, Step S104 is executed. The display control unit 312 displays the inspection assistance information on the display unit 5 in Step S104.

When the device control unit 310 determines that the information button BT10 has not been pressed in Step S111, the device control unit 310 determines whether a predetermined operation has been performed (Step S112). For example, the predetermined operation is an operation of pressing the freeze button. The predetermined operation may be an operation of hiding the information button BT10.

When the device control unit 310 determines that the predetermined operation has not been performed in Step S112, Step S111 is executed. When the device control unit 310 determines that the predetermined operation has been performed in Step S112, Step S105 is executed. At this time, the display control unit 312 may hide the information button BT10.

In the processing shown in FIG. 15, in a case in which the information button BT10 is pressed, the display control unit 312 displays the inspection assistance information on the display unit 5 in Step S104. In a case in which the information button BT10 is not pressed, the display control unit 312 does not display the inspection assistance information on the display unit 5.

In the first modified example of the first embodiment, when the information button BTU) is pressed, the endoscope device 1 displays the inspection assistance information on the display unit 5. A user can select whether to display the inspection assistance information.

### (Second Modified Example of First Embodiment)

A second modified example of the first embodiment of the present invention will be described. The inspection result storage unit 83 stores inspection result information indicating two or more inspection results. The inspection result acquisition unit 822 acquires the inspection result information indicating the two or more inspection results from the inspection result storage unit 83. The display control unit 312 displays the inspection assistance information on the display unit 5 on the basis of the priority set in advance.

For example, the priority is set in accordance with the rank of damage. The priority increases as the severity of damage increases. For example, the rank of damage is “replacement required (Reject),” “repair required (Repair),” “re-inspection required (Re-Inspect),” “no mark (No Mark),” or “no problem (Accept).” The priority is set in accordance with this order.

The priority may be set in accordance with the number of pieces of damage for each type of damage. The number indicates an occurrence ratio or occurrence number of damage. The priority increases as the number of pieces of damage increases.

The memory card 11 stores priority information indicating the type of priority. The type of priority indicates a rank of damage or the number of pieces of damage. The device control unit 310 reads the priority information from the memory card 11 via the I/F 40.

For example, after inspection assistance information corresponding to an inspection result C1 is displayed on the display unit 5, a user inputs an instruction to switch the inspection assistance information into the endoscope device 1. When the instruction is input, inspection assistance information corresponding to an inspection result C2 is displayed on the display unit 5. The priority of the inspection result C1 is higher than that of the inspection result C2. Specifically, the rank of the inspection result C1 is higher than that of the inspection result C2. Alternatively, the number of pieces of damage indicated by the inspection result Ci is greater than that of pieces of damage indicated by the inspection result C2.

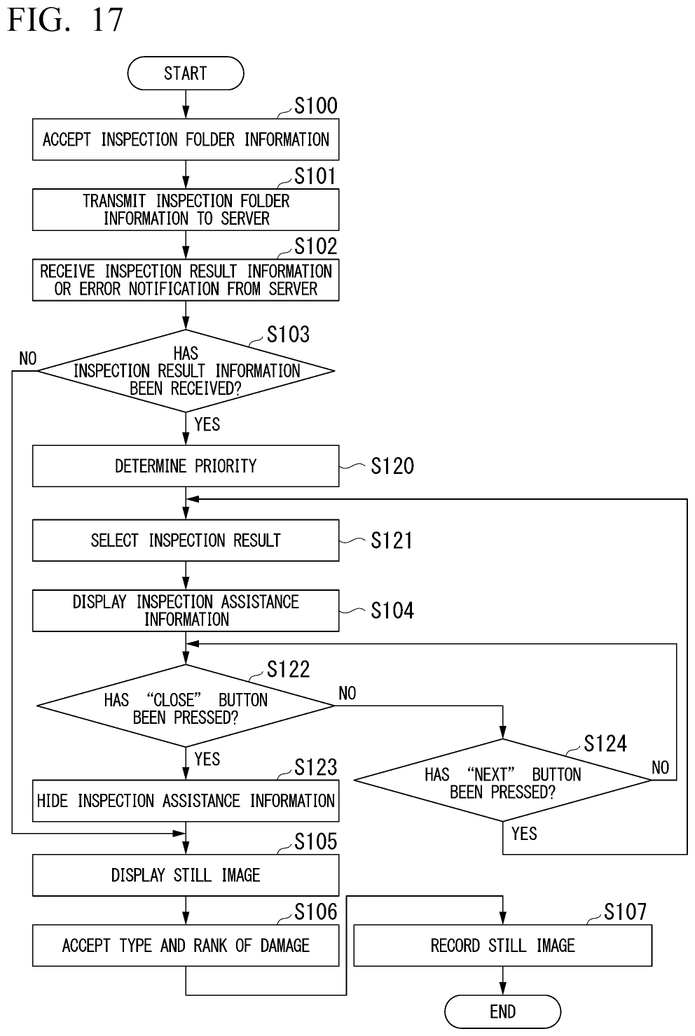

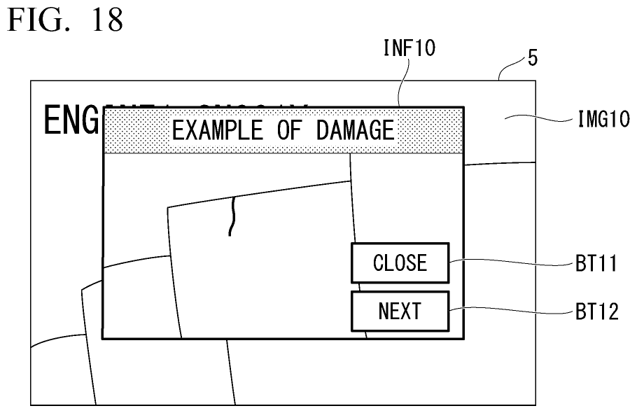

Processing executed by the endoscope device 1 will be described by using FIG. 17. FIG. 17 shows a procedure of the processing executed by the endoscope device 1. The same processing as that shown in FIG. 6 will not be described.

The inspection result acquisition unit 822 of the server 8 acquires inspection result information indicating two or more inspection results from the inspection result storage unit 83 in Step S203. For example, each of the inspection results includes information indicating the type and the rank of damage in addition to a still image of an inspection portion. The inspection result information received in Step S102 indicates the two or more inspection results.

When the device control unit 310 determines that the inspection result information has not been received in Step S103, Step S105 is executed. When the device control unit 310 determines that the inspection result information has been received in Step S103, the device control unit 310 determines the priority of the two or more inspection results by using priority information (Step S120).

In a case in which the priority information indicates the rank of damage, the device control unit 310 determines the priority of the two or more inspection results on the basis of the rank of damage. In a case in which an inspection result includes information indicating a high rank, the device control unit 310 determines that the priority of the inspection result is high. In a case in which an inspection result includes information indicating a low rank, the device control unit 310 determines that the priority of the inspection result is low.

In a case in which the priority information indicates the number of pieces of damage, the device control unit 310 determines the priority of the two or more inspection results on the basis of the number of pieces of damage. The inspection result information indicates an occurrence ratio for each type of damage or an occurrence number for each type of damage. The two or more inspection results correspond to two or more types of damage. The device control unit 310 determines the priority of the two or more types of damage. When the occurrence ratio or occurrence number of damage is great, the device control unit 310 determines that the priority of the damage is high. When the occurrence ratio or occurrence number of damage is small, the device control unit 310 determines that the priority of the damage is low.

After Step S120, the device control unit 310 selects one of the two or more inspection results (Step S121). The device control unit 310 first selects an inspection result having the highest priority.

After Step S121, the display control unit 312 displays the inspection assistance information on the display unit 5 in Step S104. At this time, the display control unit 312 displays inspection result information indicating the inspection result selected in. Step S121 as the inspection assistance information on the display unit 5. Alternatively, the display control unit 312 displays the inspection assistance information generated on the basis of the inspection result information on the display unit 5. In addition, the display control unit 312 displays a “close” button and a “next” button on the display unit 5 along with the inspection assistance information.

FIG. 18 shows inspection assistance information INF10 a live image IMG10, a “close” button BT11, and a “next” button BT12 displayed on the display unit 5. The live image IMG10 is displayed on the display unit 5, and the inspection assistance information INF10 is displayed on the live image IMG10. The inspection assistance information INF10 is an image of a damaged inspection portion. The inspection assistance information INF10 conceals part of the live image IMG10. In addition, the “close” button BT11 and the “next” button BT12 are displayed on the inspection assistance information INF10. The “close” button BT11 and the “next” button BT12 may be displayed on the live image IMG10.

A user inputs an instruction to press the “dose” button BT11 or the “next” button BT12 into the endoscope device 1 by operating the operation unit 4. Alternatively, the user inputs the instruction to press the “close” button BT11 or the “next” button BT12 into the endoscope device 1 by operating the touch panel 42. The instruction output from the operation unit 4 is input into the control unit 31 via the I/F 37. Alternatively, the instruction output from the touch panel 42 is input into the control unit 31 via the I/F 38.

After Step S104, the device control unit 310 determines whether the “close” button BT11l has been pressed (Step S122). When the instruction to press the “close” button BT11 is not output from the operation unit 4 or the touch panel 42, the device control unit 310 determines that the “close” button tall has not been pressed. When the instruction to press the “close” button BT11 is output from the operation unit 4 or the touch panel 42, the device control unit 310 determines that the “close” button BT11 has been pressed.

When the device control unit 310 determines that the “close” button BT11 has been pressed in Step S122, the display control unit 312 hides the inspection assistance information (Step S123), After Step S123, Step S105 is executed.

When the device control unit 310 determines that the “close” button BT11 has not been pressed in Step S122, the device control unit 310 determines whether the “next” button BT12 has been pressed (Step S124). When the instruction to press the “next” button BT12 is not output from the operation unit 4 or the touch panel 42, the device control unit 310 determines that the “next” button BT12 has not been pressed. When the instruction to press the “next” button BT12 is output from the operation unit 4 or the touch panel 42, the device control unit 310 determines that the “next” button BT12 has been pressed.

When the device control unit 310 determines that the “next” button BT12 has not been pressed in Step S124, Step S122 is executed. When the device control unit 310 determines that the “next” button BT12 has been pressed in Step S124, Step S121 is executed. The device control unit 310 selects, in Step S121, an inspection result having lower priority than that of the inspection result selected last.

In a case in which the priority information indicates the rank of damage, the device control unit 310 selects an inspection result of damage having a predetermined. rank (first rank) in Step S121 executed first. The display control unit 312 displays the inspection assistance information indicating the inspection result on the display unit 5 in Step S104. For example, the inspection assistance information relates to damage having the rank of “replacement required (Reject).”

The device control unit 310 selects an inspection result of damage having a second rank lower than the first rank in Step S121 executed again. The display control unit 312 displays the inspection assistance information indicating the inspection result on the display unit 5 in Step S104. For example, the inspection assistance information relates to damage having the rank of “repair required (Repair).” The type of the damage having the second rank is different from or the same as that of the damage having the first rank.

In a case in which the priority information indicates the number of pieces of damage, the device control unit 310 selects an inspection result of a predetermined number (first number) of pieces of damage in Step S121 executed first. The display control unit 312 displays the inspection assistance information indicating the inspection result on the display unit 5 in Step S104. For example, the inspection assistance information relates to damage of which the occurrence ratio or occurrence number is the greatest.

The device control unit 310 selects an inspection result of a second number of pieces of damage in Step S121 executed again. The second number is smaller than the first number. The display control unit 312 displays the inspection assistance information indicating the inspection result on the display unit 5 in Step S104. For example, the inspection assistance information relates to damage of which the occurrence ratio or occurrence number is the second greatest. The type of the second number of pieces of damage is different from that of the first number of pieces of damage.

In the first embodiment described above, the inspection assistance information may include two or more inspection results disposed in accordance with the priority. For example, when similar inspection assistance information to the inspection assistance information INF12 shown in FIG. 10 is displayed, the type of damage may be displayed at a position in accordance with the rank of the damage. For example, damage having a higher rank may be displayed on the upper side of the screen of the display unit 5, and damage having a lower rank may be displayed on the lower side of the screen of the display unit 5.

Alternatively, when similar inspection assistance information to the inspection assistance information INF14 shown in FIG. 12 is displayed, the type of damage may he displayed at a position in accordance with the occurrence ratio or occurrence number of the damage. For example, damage of which the occurrence ratio or occurrence number is great may be displayed on the upper side of the screen of the display unit 5, and damage of which the occurrence ratio or occurrence a umber is small may be displayed on the lower side of the screen of the display unit 5.

Each aspect of the present invention may include the following modified example. When two or more results are acquired in the acquisition step (Step S203), the display control unit 312 displays the inspection assistance information on the display unit 5 on the basis of the priority set in advance in the inspection assistance step (Step S104).

Each aspect of the present invention may include the following modified example. The priority is set in accordance with the state of an abnormality.

Each aspect of the present invention may include the following modified example. The display control unit 312 displays the inspection assistance information on the display unit 5 on the basis of a result indicating a state having first priority in a first inspection assistance step (Step S104). After the first inspection assistance step is executed, the display control unit 312 displays the inspection assistance information on the display unit 5 on the basis of a result indicating a state having second priority lower than the first priority in a second inspection assistance step (Step S104).

Each aspect of the present invention may include the following modified example. The priority is set in accordance with the number of abnormalities for each type of the abnormalities.

Each aspect of the present invention may include the following modified example. The display control unit 312 display inspection assistance information on the display unit 5 on the basis of a result indicating the type of an abnormality detected a first number of times in a first inspection assistance step (Step S104). After the first inspection assistance step is executed, the display control unit 312 displays the inspection assistance information on the display unit 5 (Step S104) on the basis of a result indicating the type of an abnormality detected a second number of times in a second inspection assistance step. The second number is less than the first number.

In the second modified example of the first embodiment, the endoscope device 1 displays the inspection assistance information on the display unit 5 in accordance with the priority. A user can check the inspection assistance information in order of the priority.

### (Third Modified Example of First Embodiment)

A third modified example of the first embodiment of the present invention will be described. In the third modified example of the first embodiment, the inspection folder information includes the name of an inspection portion. A user selects an inspection folder by selecting an inspection portion.

The server 8 manages a relationship between an inspection folder and an inspection portion. The communication control unit 313 of the endoscope device 1 receives information indicating the relationship from the server 8 by using the communication unit 44. The information is recorded on the memory card 11.

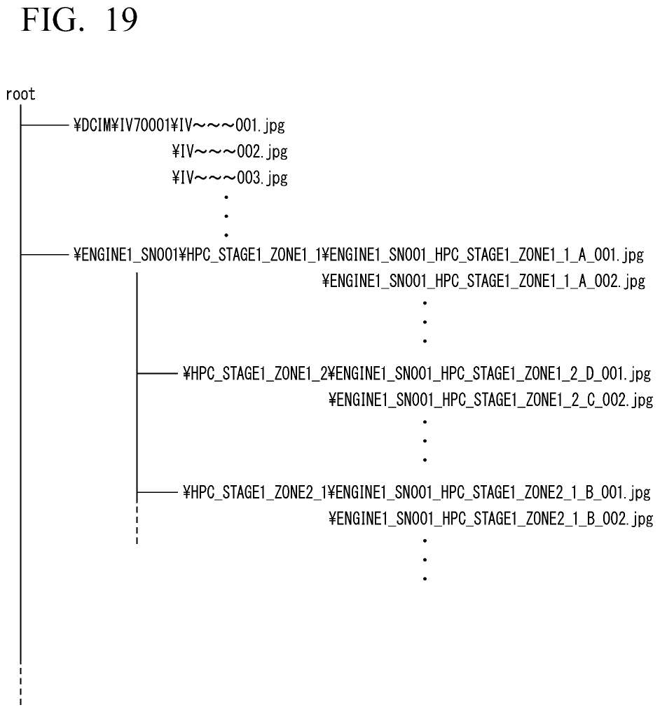

FIG. 19 shows an example of the structure of the inspection folder on the inspection result storage unit 83. In FIG. 19, each inspection folder and a file included in the inspection folder are schematically shown. An example in which the inspection folder includes two levels (classes) is shown.

A “root” folder is generated on the inspection result storage unit 83. “DCIM” folder is located under the “root” folder, and an “IV70001” folder is located under the “DCIM” folder.

An “ENGINE1_SN001.” folder is located under the “root” folder, and an “HPC_STAGE1_ZONE1_1” folder, an “HPC_STAGE1_ZONE1_2” folder, and an “HPC_STAGE1_ZONE2_1” folder are located under the “ENGINE1_SN001” folder.

A folder other than the“HPC_STAGE1_ZONE1_1,” folder, the “HPC_STAGE1_ZONE1_2” folder, or the “HPC_STAGE1_ZONE2_1” folder may be located under the “ENGINE1_SN001” folder. A folder other than the “DCIM” folder or the “ENGINE1_SN001” folder may be located under the “root” folder.

“ENGINE1” in the name of the “ENGINE1_SN001” folder indicates the name of an engine. “SN001” indicates a serial number or the like. “ENGINE1_SN001” indicates an inspection target.

A file of a still image is stored in each of the “HPC_STAGE1_ZONE1_1” folder, the “HPC_STAGE1_ZONE12” folder, and the “HPC_STAGE1_ZONE2_1” folder. The file stored in each folder consists of inspection result information. Each of “HPC_STAGE1_ZONE1_1,” “HPC_STAGE1_ZONE1_2,” and “HPC_STAGE1_ZONE2_1” indicates an inspection portion.

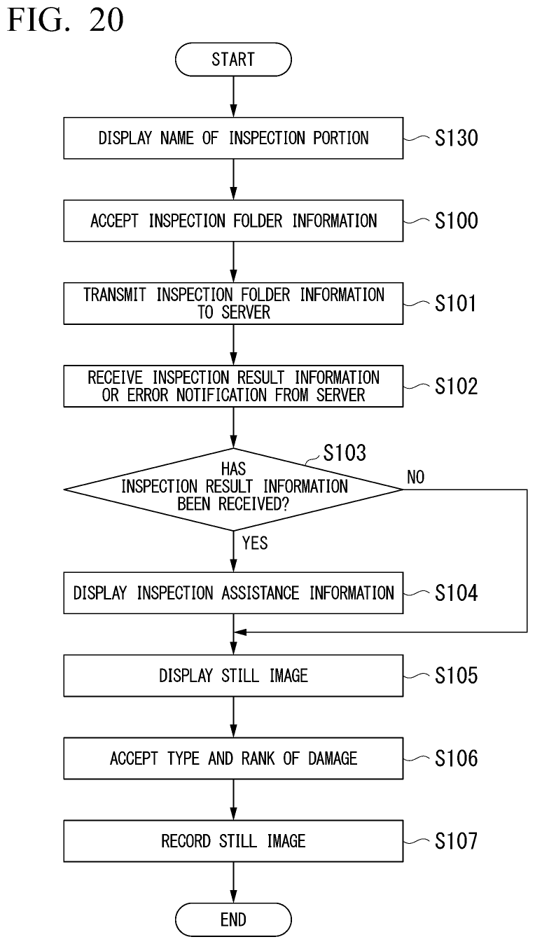

Processing executed by the endoscope device 1 will be described by using FIG. 20. FIG. 20 shows a procedure of the processing executed by the endoscope device 1. The same processing as that shown in FIG. 6 will not be described.

The display control unit 312 outputs information including a name of an inspection folder to the display unit 5 via the I/F 39. In this way, the display control unit 312 displays the information on the display unit 5 (Step S130). The name of the inspection folder includes a name of an inspection portion. After Step S130, Step S100 is executed.

The display control unit 312 may display a list of names of inspection folders on the display unit 5. The display control unit 312 may display a live image of an inspection portion and a name of an inspection folder on the display unit 5.

A user selects a name of an inspection folder by operating the joystick of the operation unit 4. In this way, the user inputs the name into the endoscope device 1. The acceptance unit 311 accepts the name in Step S100. The acceptance unit 311 generates inspection folder information corresponding to the name of the inspection portion by using the information indicating the relationship between the inspection folder and the inspection portion.

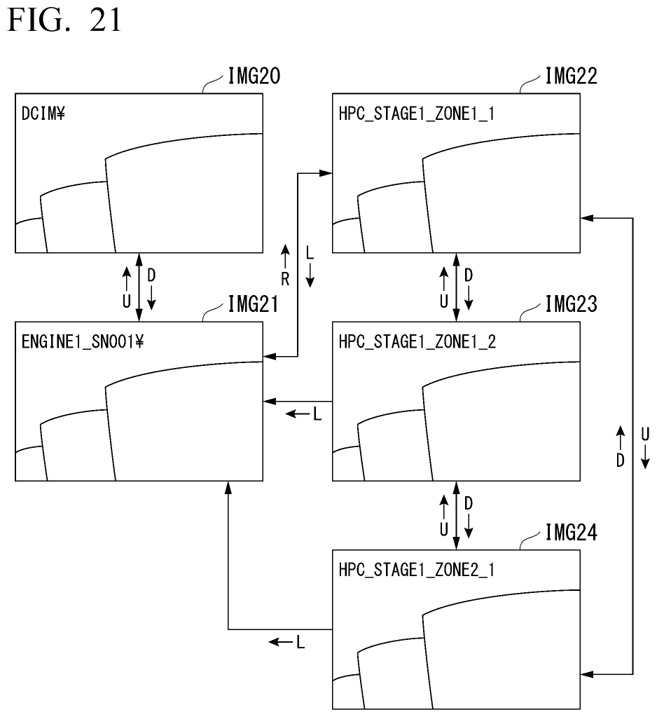

FIG. 21 shows an example of a change of the inspection folder information displayed on the display unit 5 in Step S130. A live image of an inspection target and a name of an inspection folder are displayed. Immediately after the power source of the endoscope device 1 is turned on, an image IMG20 is displayed on the display unit 5. The image IMG 20 includes a character string indicating the name of the “DCIM” folder under the “root” folder.

A user can select an inspection folder by operating the joystick of the operation unit 4. The user tilts the joystick either upward (U), downward (D), left (L), or right (R). Any one of a plurality of inspection folders is selected in accordance with the direction of the joystick. When the user tilts the joystick in a predetermined direction and a button of the operation unit 4 is pressed, inspection folder information indicating the selected inspection folder is input into the endoscope device 1.

When the image IMG20 is displayed, the user tilts the joystick, for example, downward (D), In this way, the “ENGINE1_SN001” folder under the “DCIM” folder is selected, and an image IMG21 is displayed on the display unit 5. The image IMG 21 includes a character string indicating the name of the “ENGINE1_SN001” folder. The character string includes a name of an inspection target.

When the image IMG21 is displayed, the user tilts the joystick, for example, upward (U). In this way, the “DCIM” folder above the “ENGINE1_SN001” folder is selected, and the image IMG20 is displayed on the display unit 5.

When the image IMG21 is displayed, the user tilts the joystick, for example, right (R). In this way, the “HPC_STAGE1_ZONE1_1” folder of the three inspection folders under the “ENGINE1_SN001” folder is selected, and an image IMG22 is displayed on the display unit 5. The image IMG 22 includes a character string indicating the name of the “HPC_STAGE1_ZONE1_1” folder. The character string includes a name of an inspection portion.

When the image IMG22 is displayed, the user tilts the joystick, for example, left (L). In this way, the “ENGINE1_SN001” folder above the “HPC_STAGE1_ZONE1_1” folder is selected, and the image IMG21 is displayed on the display unit 5.

When the image IMG22 is displayed, the user tilts the joystick, for example, downward (D). In this way, the “HPC_STAGE1_ZONE1_2” folder having the same level as that of the “HPC_STAGE1_ZONE1_1” folder is selected, and an image IMG23 is displayed on the display unit 5. The image IMG 23 includes a character string indicating the name of the “HPC_STAGE1_ZONE1_2” folder. The character string includes a name of an inspection portion.

When the image IMG23 is displayed, the user tilts the joystick, for example, upward (U). In this way, the “HPC_STAGE1_ZONE1_1” folder having the same level as that of the “HPC_STAGE1_ZONE1_2” folder is selected, and the image IMG22 is displayed on the display unit 5.

When the image IMG23 is displayed, the user tilts the joystick, for example, downward (I)). In this way; the “HPC_STAGE1_ZONE2_1” folder having the same level as that of the “HPC_STAGE_ZONE1_2” folder is selected, and an image IMG24 is displayed on the display unit 5. The image IMG 24 includes a character string indicating the name of the “HPC_STAGE1_ZONE2_1” folder. The character string includes a name of an inspection portion.

When the image IMG24 is displayed, the user tilts the joystick, for example, upward (U). In this way, the “HPC_STAGE1_ZONE1_2” folder having the same level as that of the “HPC_STAGE1_ZONE2_1” folder is selected, and the image IMG23 is displayed on the display unit 5.

When the image IMG22 is displayed, the user tilts the joystick, for example, upward (U). In this way, the “HPC_STAGE1_ZONE2_1” folder having the same level as that of the “HPC_STAGE1_ZONE1_1” folder selected, and the image IMG24 is displayed on the display unit 5.

When the image IMG24 is displayed, the user tilts the joystick, for example, downward (D). In this way, the “HPC_STAGE1_ZONE1_1” folder having the same level as that of the “HPC_STAGE1_ZONE2 1” folder is selected, and the image IMG22 is displayed on the display unit 5.

When the image IMG23 or the image IMG24 is displayed, the user tilts the joystick, for example, left (L). In this way, the “ENGINE1_SN001” folder above the “HPC_STAGE1_ZONE1_2” folder or the “HPC_STAGE1_ZONE2_1” folder is selected, and the image IMG21 is displayed on the display unit 5.

The endoscope device 1 may include a code reader. The endoscope device 1 may read a two-dimensional code by using the code reader. The two-dimensional code is attached on an inspection portion and includes information of the inspection portion. The endoscope device 1 may generate inspection folder information corresponding to the inspection portion indicated by the two-dimensional code.

Each aspect of the present invention may include the following modified example. Before the acceptance step (Step S100) is executed, the display control unit 312 displays inspection portion information, that is, a nine of an inspection portion on the display unit 5 in an information display step (Step S130).

In the third modified example of the first embodiment, a user can input the inspection folder information into the endoscope device 1 by selecting the name of the inspection portion displayed on the display unit 5.

### (Fourth Modified Example of First Embodiment)

A fourth modified example of the first embodiment of the present invention will be described. In the fourth modified example of the first embodiment, the display control unit 312 displays an image of an inspection target on the display unit 5, and displays a name of an inspection portion on the image. A user selects an inspection folder by selecting an inspection portion. The memory card 11 stores information indicating a relationship between the inspection folder and the inspection portion in advance.

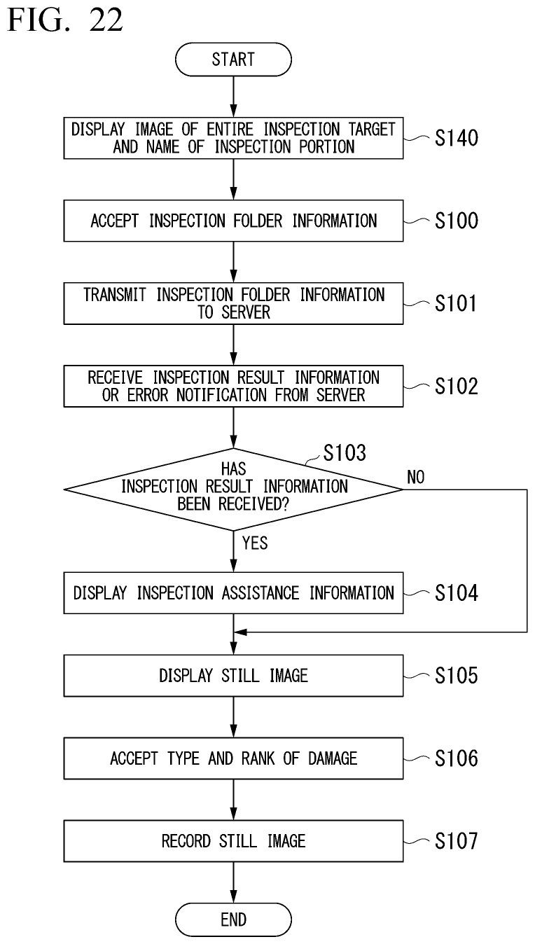

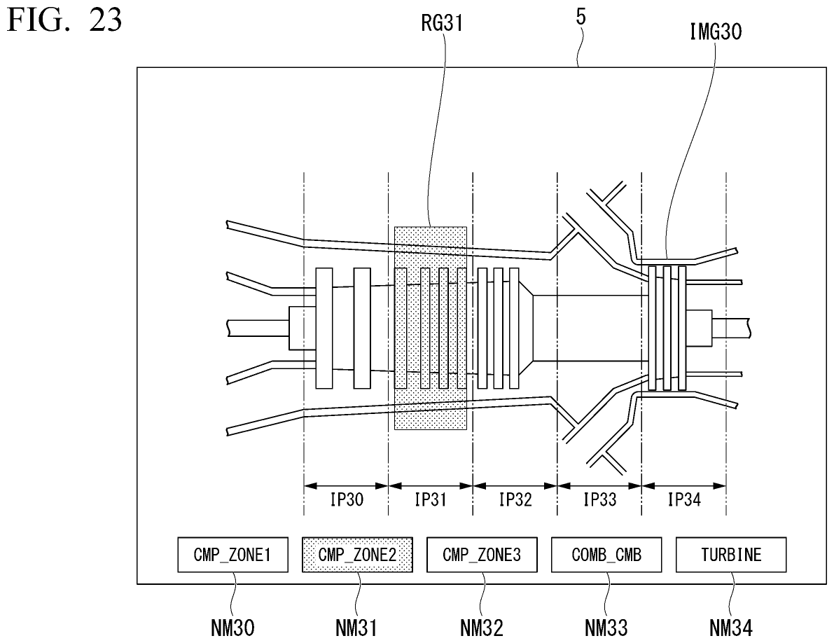

Processing executed by the endoscope device 1 will be described by using FIG. 22. FIG. 22 shows a procedure of the processing executed by the endoscope device 1. The same processing as that shown in FIG. 6 will not be described.

The display control unit 312. outputs an image including both an image of the entire inspection target and a name of an inspection portion to the display unit 5 via the I/F 39. In this way, the display control unit 312 displays the image on the display unit 5 (Step S140). After Step S140, Step S100 is executed.

A user selects a name of an inspection portion by operating the touch panel 42. In this way, the user inputs the name into the endoscope device 1. The acceptance unit 311 accepts the name in Step S100. The acceptance unit 311 generates inspection folder information corresponding to the name of the inspection portion by using the information indicating the relationship between the inspection folder and the inspection portion.

FIG. 23 shows an example of an image displayed on the display unit 5 in Step S140. An image IMG30, a name NM30, a name NM31, a name NM32, a name NM33, and a name NM34 are displayed on the display unit 5. The image IMG30 is an image of the entire inspection target. Each of the names NM30 to NM34 indicates a name of an inspection portion.

A position of each of an inspection portion IP30, an inspection portion IP31, an inspection portion IP32, an inspection portion IP33, and an inspection portion IP34 is displayed on the image IMG30. The inspection portion IP30 has the name NM30, the inspection portion IP31 has the name NM31, the inspection portion IP32 has the name NM32, the inspection portion IP33 has the name NM33, and the inspection portion IP34 has the name NM34.

In the example shown in FIG. 23, a user selects the name NM31. The color of the name NM31 is changed, and a region RG31 of the inspection portion IP31 having the name NM31 is displayed on the image IMG30. The user can cancel selection of the name and can select another name by operating the joystick. For example, lien the user selects the name NM32, the color of the name NM32 is changed and a region of the inspection portion IP32 having the name NM32 is displayed on the image IMG30. At this time, the color of the name NM31 returns to its original color, and the region RG31 of the inspection portion IP31 is hidden. Furthermore, the user inputs the inspection folder information corresponding to the inspection portion having the selected name into the endoscope device 1 by operating the joystick.

Alternatively, when the user selects any one of the names NM30 to NM34 by operating e touch panel 42, the inspection folder information corresponding he inspection portion having the selected name is input into the endoscope device 1.

Each aspect of the present invention array include the folio modified example. The display control unit 312 displays an age of an inspection target on the display unit 5 and displays inspection portion information, that is, a name of an inspection portion on the image in an information display step (Step S140).

In the fourth modified example of the first embodiment, a user can check the position of the inspection portion in the entire inspection target. The user can input the inspection folder information into the endoscope device 1 by selecting the name of the inspection portion displayed on the display unit 5.

### (Fifth Modified Example of First Embodiment)

A fifth modified example of the first embodiment of the present invention be described. In the fifth modified example of the first embodiment. the display control unit 312 displays an image of an inspection target on the display unit 5, and displays a name of an inspection portion on the image. In addition, the display control unit 312 displays information related to the number of pieces of damage on the display unit 5. The number of pieces of damage is an occurrence ratio or occurrence number of the damage. A user selects an inspection folder by selecting an inspection portion. The memory card 11 stores information indicating a relationship between the inspection. folder and the inspection portion in advance.

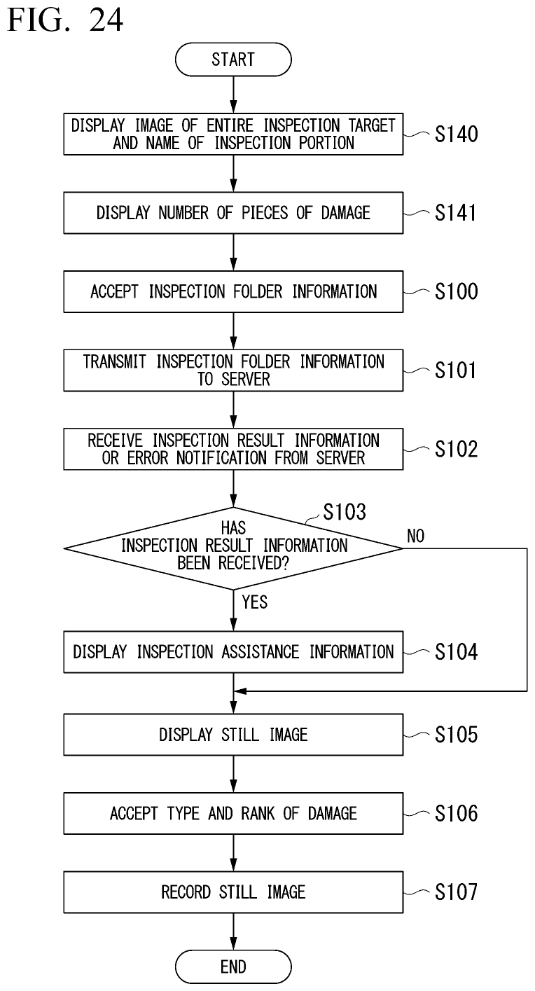

Processing executed by the endoscope device 1 will be described by using FIG. 24. FIG. 24 shows a procedure of the processing executed by the endoscope device 1. The same processing as that shown in FIG. 22 will not be described.

After Step S140, the display control 312 outputs an image including information related to the number of pieces of damage to the display unit 5 via the I/F 39.

In this way, the display control it 312 displays the image on the display unit 5 (Step S141). After step S141, Step S100 is executed.

A user selects a name of an inspection portion by operating the joystick or the touch panel 42. In this way, the user inputs the name into the endoscope device 1. The acceptance unit 311 accepts the name in Step S100. The acceptance unit 311 generates inspection folder information corresponding to the name of the inspection portion by using the information indicating the relationship between the inspection folder and the inspection portion.

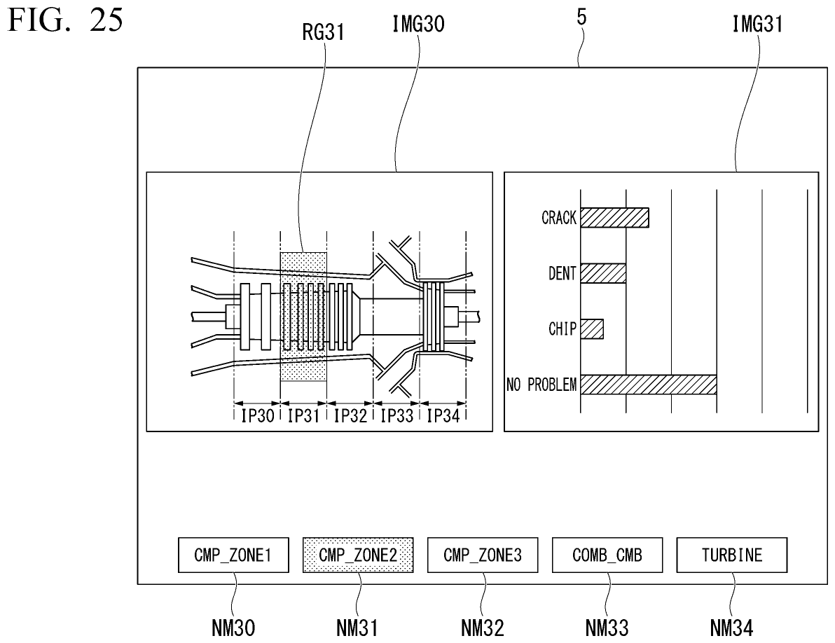

FIG. 25 shows an example of an image displayed on the display unit 5 in Step S141. An image IMG30, an image IMG31, a name NM30, a name NM31 a name NM32, a name NM33, and a name NM34 are displayed on the display unit 5. The image IMG30 is an image of the entire inspection target. Each of the names NM30 to NM34 indicates a name of an inspection portion.

A position of each of an inspection portion IP30, an inspection portion IP31, an inspection portion IP32, an inspection portion IP33, and an inspection portion IP34 is displayed on the image IMG30. A relationship between the inspection portions IP30 to IP34 and the names NM30 to NM34 is the same as that shown in FIG. 23. The image IMG31 indicates an occurrence ratio or occurrence number for each type of damage previously detected in the inspection portion.

In the example shown in FIG. 25, a user selects the name NM31. The color of the name NM31 is changed, and a region RG31 of the inspection portion IP31 having the name NM31 is displayed on the image IMG30. In addition, the image IMG31 related to the inspection portion corresponding to the name NM31 is displayed. The image IMG31. Indicates an occurrence ratio or occurrence number of damage previously detected in the inspection portion corresponding to the name NM31.

The user can cancel selection of the name and can select another name by operating the joystick. For example, when the user selects the name NM32, the color of the name NM32 is changed and a region of the inspection portion IP32 having the name NM32 is displayed on the age IMG30. At this time, the color of the name NM31 returns to its original color, and the region RG31 of the inspection portion IP31 is hidden. In addition, the image IMG31 is hidden, and an image related to the inspection portion corresponding to the name NM32 is displayed in place of the image IMG31. Furthermore, the user inputs the inspection folder information corresponding to the inspection portion having the selected name into the endoscope device 1 by operating the joystick.

Alternatively, when the user selects any one of the names NM30 to NM34 by operating the touch panel 42, the inspection folder information corresponding to the inspection portion having the selected name is input into the endoscope device 1.

In the fifth modified example of the first embodiment, a user can check the position of the inspection portion in the entire inspection target. In addition, the user can check the number of abnormalities in the inspection portion. The user can input the inspection folder information into the endoscope device 1 by selecting the name of the inspection portion displayed on the display unit 5.

### (Sixth Modified Example of First Embodiment)

A sixth modified example of the first embodiment of the present invention will be described. In the sixth modified example of the first embodiment, the memory card 11 stores an inspection plan in advance. The inspection plan includes information related to an inspection portion scheduled to be inspected in an inspection that has not been performed yet. For example, the information includes the name of the inspection portion, the position of the inspection portion, and order of an inspection. The display control unit 312 displays information of the inspection portion on the display unit 5 on the basis of the inspection plan. The memory card 11 stores information indicating a relationship between the inspection folder and the inspection portion in advance.

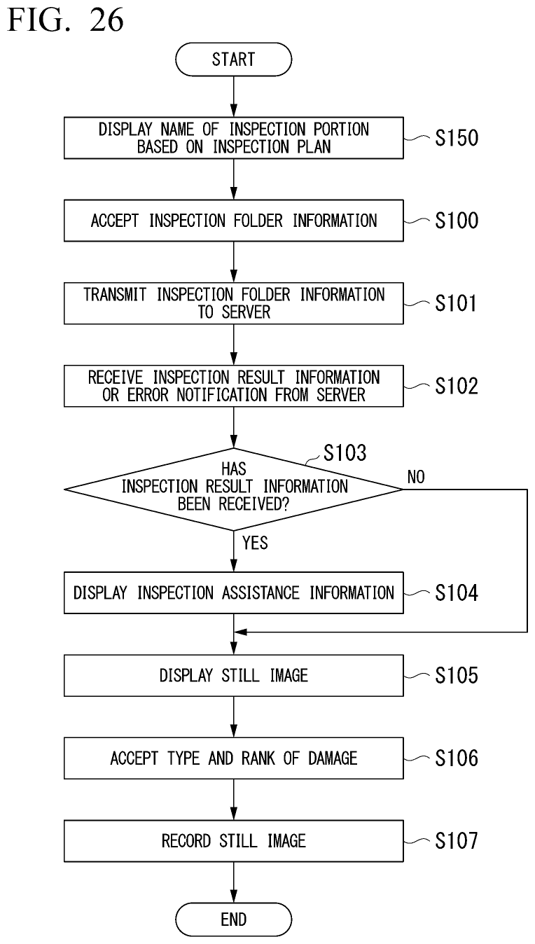

Processing executed by tine endoscope device 1 will be described by using FIG. 26. FIG. 26 shows a procedure of the processing executed by the endoscope device 1. The same processing as that shown in FIG. 6 will not be described.

The device control unit 310 reads the inspection plan from the the memory card 11 via the I/F 40. The display control unit 312 refers to the inspection plan and extracts information of the inspection portion from the inspection plan. The display control unit 312 outputs the extracted information to the display unit 5 via the I/F 39. In this way, the display control unit 312 displays the information on the display unit 5 (Step S150). For example, the display control unit 312 displays a list of names of inspection portions on the display unit 5. After Step S150, Step S100 is executed.

A user selects a name of an inspection portion by operating the joystick or the touch panel 42. In this way, the user inputs the name into the endoscope device 1. The acceptance unit 311 accepts the name in Step S100. The acceptance unit 311 generates inspection folder information corresponding to the name of the inspection portion by using the information indicating the relationship between the inspection folder and the inspection portion.

Each aspect of the present invention may include the following modified example. The display control unit 312 refers to an inspection plan and displays inspection portion information on the display unit 5 on the basis of the inspection plan an information display step (Step S150). The inspection plan includes the inspection portion information related to an inspection portion scheduled to be inspected.

In the sixth modified example of the first embodiment, a user can input the inspection folder information into the endoscope device 1 by selecting the name of the inspection portion displayed on the display unit 5.

### (Second Embodiment)

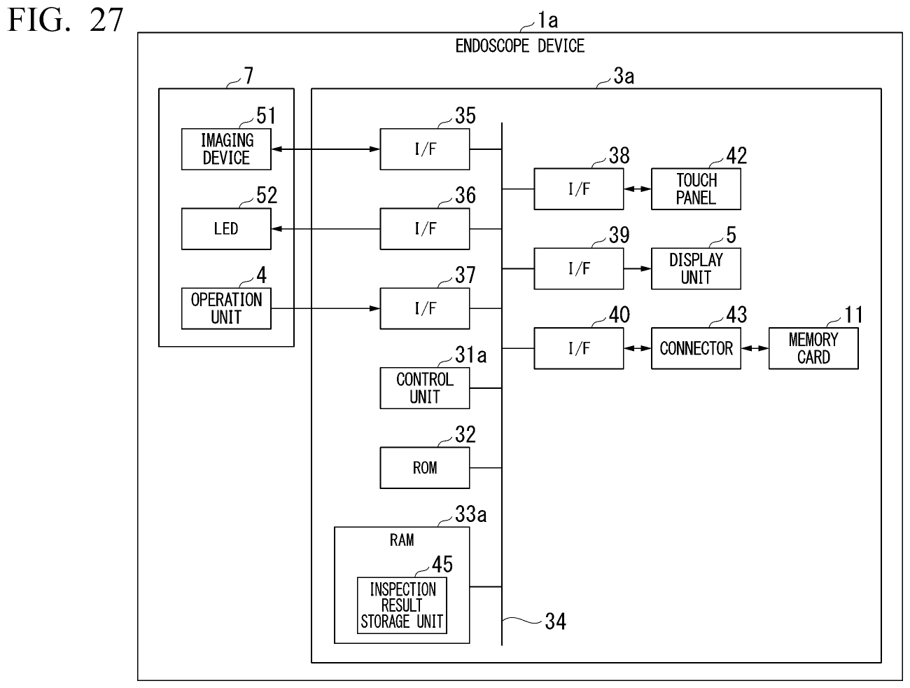

A second embodiment of the present invention will be described. The endoscope device 1 shown in FIG. 3 is changed to an endoscope device 1a shown in FIG. 27. FIG. 27 shows a configuration of the endoscope device 1a. The same configuration as that shown in FIG. 3 will not be described. In the second embodiment, the server 8 does not need to be used.

The main body unit 3 shown in FIG. 3 is changed to a main body unit 3a shown in FIG. 27. In the main body unit 3a, the control unit 31 shown in FIG. 3 is changed to a control unit 31a shown in FIG. 27. In the main body unit 3a, the RAM 33 shown in FIG. 3 is changed to a RAM 33a. The RAM 33a includes an inspection result storage unit 45. The inspection result storage unit 45 stores inspection result information indicating an inspection result. The main body unit 3a does not include I/F 41 or the. communication unit 44 shown in FIG. 3.

The control unit 31a is constituted by at least one of a processor and a logic circuit. The control unit 31a pray include one or a plurality of processors. The control unit 31a may elude one or a plurality of logic circuits.

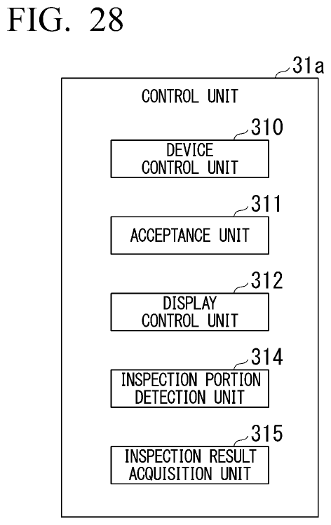

FIG. 28 shows a configuration of the control unit 31a. The control unit 31a includes a device control unit 310, an acceptance unit 311, a display control unit 312, an inspection portion detection unit 314, and an inspection result acquisition unit 315. The same configuration as that shown in FIG. 4 will not be described.

The control unit 31a does not include the communication control unit 313 shown in FIG. 4. The inspection portion detection unit 314 detects an inspection portion corresponding to the inspection folder indicated by the inspection folder information. The inspection result acquisition unit 315 acquires the inspection result information of the inspection portion detected by the inspection portion detection unit 314 from the inspection result storage unit 45.

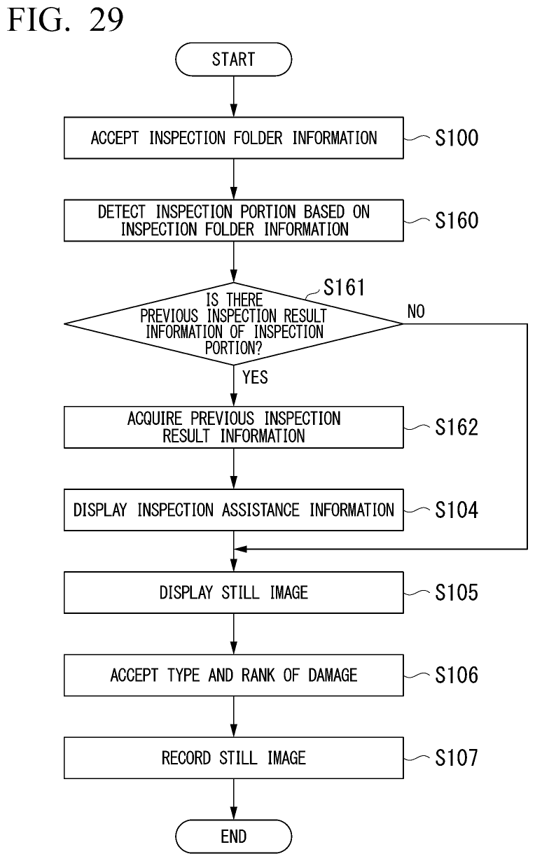

Processing executed by the endoscope device la will be described by using FIG. 29. FIG. 29 shows a procedure of the processing executed by the endoscope device la, The same processing as that shown in FIG. 6 will not be described.

After Step S100, the inspection portion detection unit 314 detects an inspection portion on the basis of the inspection folder information input in Step S100 (Step S160). Step S160 is similar to Step S201 shown in FIG. 7.

After Step S160, the inspection result acquisition unit 315 searches the inspection result storage unit 45 for the inspection result information of the inspection portion detected in Step S160. The inspection result acquisition unit 315 determines whether there is previous inspection result information of the inspection portion on the basis of the result of the search (Step S161). Step S161 is similar to Step S202 shown in FIG. 7.

In a case in which the inspection result acquisition unit 315 determines that there is no previous inspection result information of the inspection portion in Step S161, Step S105 is executed. in a case in which the inspection result acquisition unit 315 determines that there is the previous inspection result information of the inspection portion in Step S161, the inspection result acquisition unit 315 acquires the inspection result information from the inspection result storage unit 45 (Step S162). Step S162 is similar to Step S203 shown in FIG. 7. After Step S162, Step S104 is executed.

The processing shown in FIG. 29 may include Steps S110 to S112 shown in FIG. 15. The processing shown in FIG. 29 may include Steps S120 to S124 shown in FIG. 17. The processing shown in FIG. 29 may include Step S130 shown in FIG. 20. The processing shown in FIG. 29 may include Step S140 shown in FIG. 22. The processing shown in FIG. 29 may include Step S140 and Step S141 shown in FIG. 24. The processing shown in FIG. 29 may include Step S150 shown in FIG. 26.

An inspection assistance device (endoscope device 1a) according to each aspect of the present invention includes the acceptance unit 311, the inspection result acquisition unit 315, and the display control unit 312 (an inspection assistance unit and an image display unit). The acceptance unit 311 accepts inspection portion information related to an inspection portion included in at least part of an inspection target. The inspection result acquisition unit 315 connects to a storage medium (inspection result storage unit 45) storing a result of a previous inspection and acquires the result of the inspection of the inspection portion corresponding to the inspection portion information from the storage medium. The display control unit 312 displays inspection assistance information on the display unit 5 on the basis of the result acquired by the inspection result acquisition unit 315. The inspection assistance information notifies a user of information related to an abnormality of the inspection portion detected in the previous inspection. After the inspection assistance information is displayed, the display control unit 312 displays an image of the inspection portion on the display unit 5.

In the second embodiment, the endoscope device la displays the inspection assistance information on the display unit 5. Therefore, the endoscope device 1a can reduce a possibility that a user overlooks an abnormality or mistakes evaluation of the abnormality.

While preferred embodiments of the invention have been described and shown above, it should be understood that these are examples of the invention and are not to be considered as limiting. Additions, omissions, substitutions, and other modifications can be made without departing from the spirit or scope of the present invention. Accordingly, the invention is not to be considered as being limited by the foregoing description, quad is only limited by the scope of the appended claims.

What is claimed is:

1. An inspection assistance method executed by a processor, comprising:
accepting inspection portion information related to an inspection portion included in at least part, of an inspection target;
connecting to a storage medium storing a result of a previous inspection;
acquiring the result of the inspection portion corresponding to the inspection portion information from the storage medium;
displaying inspection assistance information on a display on the basis of the acquired result,
wherein the inspection assistance information notifies a user of information related to an abnormality of the inspection portion detected in the previous inspection; and
displaying an image of the inspection portion on the display after the inspection assistance information is displayed.

2. The inspection assistance method according to claim 1,
wherein, when two or more of the results are acquired, the displaying of the inspection assistance information comprises displaying the inspection assistance information on the display on the basis of priority set in advance.

3. The inspection assistance method according to claim 2,
wherein the priority is set in accordance with a state of the abnormality.

4. The inspection assistance method according to claim 3,
wherein the displaying of the inspection assistance information comprises:
displaying the inspection assistance information on the display on the basis of the result indicating the state having first priority; and
displaying the inspection assistance information on the display on the basis of the result indicating the state having second priority lower than the first priority after the inspection assistance information is displayed on the basis of the result indicating the state having the first priority.

5. The inspection assistance method according to claim 2,
wherein the priority is set in accordance with the number of the abnormalities for each type of the abnormalities.

6. The inspection assistance method according to claim 5,
wherein the displaying of the inspection assistance information comprises:
displaying the inspection assistance information on the display on the basis of the result indicating the type of the abnormality detected a first number of times; and
displaying the inspection assistance information on the display on the basis of the result indicating the type of the abnormality detected a second number of times after the inspection assistance information is displayed on the basis of the result indicating the type of the abnormality detected the first number of times,
wherein the second number is less than the first number.

7. The inspection assistance method according to claim 1, further comprising displaying the inspection portion information on the display before the inspection portion information is accepted.

8. The inspection assistance method according to claim 7,
wherein the displaying of the inspection portion information comprises:
referring to an inspection plan including the inspection portion information related to the inspection portion scheduled to be inspected; and
displaying the inspection portion information on the display on the basis of the inspection plan.

9. The inspection assistance method according to claim 7,
wherein the displaying of the inspection portion information comprises:
displaying the image of the inspection target on the display; and
displaying the inspection portion information on the image.

10. The inspection assistance method according to claim 1,
wherein the inspection portion information indicates a folder of the storage medium in which the result saved.
wherein the folder is prepared for each inspection portion and is associated with the inspection portion, and
wherein the acquiring of the result of the inspection portion comprises acquiring the result of the inspection portion from the folder indicated by the inspection portion information.

11. Th inspection assistance method according to claim 1,
wherein the inspection assistance information includes an image of the abnormality detected in a previous inspection.

12. The inspection assistance method according to claim 1,
wherein the inspection assistance information indicates the number of the abnormalities for each type of the abnormalities.

13. The inspection assistance method according to claim 1,
wherein the inspection assistance information indicates a proportion of the number of the abnormalities for each type of the abnormalities to the number of all abnormalities previously detected in the inspection portion.

14. The inspection assistance method according to claim 1,
wherein the acquiring of the result of the inspection portion comprises acquiring the result of an inspection previously performed for the same object as the inspection target or for an object of the same type as a type of the inspection target.

15. The inspection assistance method according to claim 1,
wherein the acquiring of the result of the inspection portion comprises:
searching for the result of the inspection portion in the same object as the inspection target, and
acquiring the result of the inspection portion in an object of the same type as a type of the inspection target in a case in which the result of the inspection portion is not acquired.

16. The inspection assistance method according to claim 1,
wherein the displaying of the inspection assistance information comprises displaying one of two or more sequentially generated images of the inspection portion the display.

17. The inspection assistance method according to claim 1, further comprising, after the inspection assistance information is displayed, hiding the inspection assistance information or reducing a size of a region of the inspection assistance information displayed on the display when movement of an insertion unit inserted into the inspection target is detected.

18. The inspection assistance method according to claim 1,
wherein the displaying of the inspection assistance information comprises:
displaying the inspection assistance information on the image of the inspection portion, and
after the inspection assistance information is displayed, changing a transmittance of the inspection assistance information when movement of an insertion unit inserted into the inspection target is detected.

19. An inspection assistance device, comprising a processor configured to:
accept inspection portion information related to an inspection portion included in at least part of an inspection target;
connect to a storage medium storing a result of a previous inspection;
acquire the result of the inspection portion corresponding to the inspection portion information from the storage medium;
display inspection assistance information on a display on the basis of acquired result,
wherein the inspection assistance information notifies a user of information related to an abnormality of the inspection portion detected in the previous inspection; and
display an image of the inspection portion on the display after the inspection assistance information is displayed.

20. An inspection assistance system, comprising:
an inspection assistance device comprising:
a first communicator configured to transmit inspection portion information related to an inspection portion included in at least part of an inspection target to a server and receive a result of a previous inspection of the inspection portion from the server; and
a first processor configured to:
accept the inspection portion information;
display inspection assistance information on a display on the basis of the result received by the first communicator,
wherein the inspection assistance information notifies a user of information related to an abnormality of the inspection portion detected in the previous inspection; and
display an image of the inspection portion on the display after the inspection assistance information is displayed; and
the server comprising:
a second processor configured to connect to a storage medium storing a result of a previous inspection and acquire the result of the inspection of the inspection portion corresponding to the inspection portion information from the storage medium; and
a second communicator configured to receive the inspection portion information from the inspection assistance device and transmit the result acquired by the second processor to the inspection assistance device.

21. A non-transitory computer-readable recording medium storing a program causing a computer to execute:
accepting inspection portion information related to an inspection portion included in at least part of an inspection target;
connecting to a storage medium storing a result of a previous inspection;
acquiring the result of the inspection portion corresponding to the inspection portion information from the storage medium;
displaying inspection assistance information on a display on the basis of the acquired result,
wherein the inspection assistance information notifies a user of information related to an abnormality of the inspection portion detected in the previous inspection; and
displaying an image of the inspection portion on the display after the inspection assistance information is displayed.
